(19) United States
(12) Patent Application Publication
(10) Pub. No.: US 2015/0233926 A1
(43) Pub. Date: Aug. 20, 2015

(54) METHOD FOR SPECIFICALLY DETECTING A MATRIX METALLOPROTEINASE (MMP) WHICH IS ONLY OF INTEREST IN THE ACTIVE FORM THEREOF, IN A SAMPLE

(71) Applicant: COMMISSARIAT A L'ENERGIE ATOMIQUE ET AUX ENERGIES ALTERNATIVES, Paris (FR)

(72) Inventors: Sarah Bregant, Paris (FR); Vincent Dive, Palaiseau (FR); Marie-Claire Nevers, Villebon Sur Yvette (FR)

(21) Appl. No.: 14/429,600

(22) PCT Filed: Sep. 24, 2013
(86) PCT No.: PCT/FR2013/052224

§ 371 Date: Mar. 19, 2015

(30) Foreign Application Priority Data
Sep. 25, 2012 (FR) 1258996

(51) Int. Cl. G01N 33/573

(52) U.S. Cl. CPC G01N 33/573; G01N 2333/96494; G01N 2458/00

(57) ABSTRACT

The present invention relates to a method that allows to detect the active form of a specific matrix metalloproteinase and its use in diagnostic methods.

The present invention relates to a method for specifically detecting a matrix metalloproteinase (MMP).

### BACKGROUND OF THE INVENTION

MMPs are capable of collectively degrading all components of the extracellular matrix (ECM). ECM degradation, in addition to permitting cell migration, leads to the release of signaling molecules originally bound to the ECM such as chemokines, cytokines, or growth factors. MMPs also contribute to the activation of signaling molecules.

The activity of MMPs in tissue remodeling is tightly controlled in vivo by natural inhibitors known as TIMPs (tissue inhibitors of metalloproteinases) or by latent forms that are the proforms of MMPs. Indeed, it is possible for an active form of MMPs whose active site is unbound and is therefore capable of interacting with its natural substrates, an inactive proform of MMPs whose active site is occupied by the pro-peptide, and an inhibited form of MMPs whose active site is occupied by the natural inhibitor, to all coexist.

The development and progression of diseases is accompanied by deregulation of this activity, associated with the presence of active forms capable of proteolysis. The diseases concerned are cancer (several biomarker MMPs, specifically MMP-2 and MMP-9), viral infections (MMP-9, MMP-2), osteoarthritis and rheumatoid arthritis (MMP-13), Dupuytren's contracture (MMP-2, MMP-14), atherosclerosis (MMP-12), or respiratory diseases with inflammatory components such as chronic obstructive pulmonary disease (COPD) (MMP-12), emphysema, and asthma (MMP8). MMPs also have a role in neurodegenerative diseases: multiple sclerosis (MMP-3, MMP-8, MMP-9), myasthenia gravis (MMP-2, MMP-3, MMP-9), stroke (MMP-9, MMP-3), amyotrophic lateral sclerosis (MMP-1 and MMP-2), Alzheimer's disease (MMP-3, MMP-9, and MMP-10).

The active forms of MMPs therefore represent markers of these diseases, and their detection poses a true diagnostic challenge which the invention described in this patent proposes to address.

Indeed, although many MMP detection methods have been described, there is no satisfactory method for detecting a particular MMP, and specifically its active form which means excluding the inactive forms (proforms and inhibited forms).

Through their measurement of messenger RNA, transcriptome analyses only tell us about the amount of mRNA encoding the protein existing in fluids in various forms at the end. Measurement of mRNA transcripts does not provide information about the proportion of active forms.

Methods based on synthetic substrates of MMPs pose other problems. Most of the kits sold using substrates to detect MMPs involve a step of activating the enzyme itself by adding a mercuric salt which rules out any specific measure of the presence of an initially active form. Most importantly, the use of substrates provides no information on the identity of the MMP involved in cleaving the substrate as there is no high specificity of synthetic substrates between the different MMPs. These methods are therefore not specific for a particular MMP.

All techniques based on electrophoresis use a partial or complete denaturation step that prevents distinguishing between an active enzyme form and an enzyme form whose activity is initially controlled by the presence of a natural inhibitor blocking the active site. In fact, the necessary denaturation step in these techniques eliminates the existence of inactive MMPs which are then analyzed as the active form. In addition, the use of antibodies in the Western blot technique remains little sensitive to proteins present in very low concentrations in the samples. These techniques therefore detect both the inactivated and activated forms, and are not very sensitive.

The zymography technique, highly specific for the detection of MMP2 and MMP9 (also known as gelatinases), also suffers from the disadvantage of a step of partial denaturation of the sample, which is necessary for its implementation. In addition, the routine use of electrophoresis limits the total amount of sample analyzed, due to migration methodology constraints which limit the amount of the analyte of interest in the analysis.

ELISA tests, some of which are commercially available for the detection of MMP12, use a set of complementary antibodies that do not allow distinguishing between active forms, proforms, and inactive forms, thereby preventing access to the functional information of the protein.

Because of all these elements, research has been conducted concerning specific detection of active forms of the MMPs involved in diseases through their proteolytic activity. Methods previously developed for this purpose, including activity-based probes, have disadvantages related to a lack of sensitivity or specificity, particularly when considering complex biological samples or tissue samples. Here again, the limitations are due to methodological constraints on sample handling.

The only example in the literature that is described as meeting the requirements necessary for detection of MMPs in active form relates to the detection of human MMP12, and uses a system that combines the use of an antibody and a fluorescent substrate (LaPan et al, BMC Pulmonary Medicine 2010, 10:40). However, no data on the ability of such systems to exclude the detection of MMP12 initially inactivated by an inhibitor is provided. However, this method comprises a preliminary washing step between the binding of the protein to the antibody and the addition of the substrate, a step which could remove natural inhibitors (TIMPs) bound to the MMP12. Therefore, this method can distinguish between the proform and active form in a biological sample, but cannot distinguish between active forms where the active site is unbound and those where the active site is initially occupied by an inhibitor. Moreover, such an approach can only be used in the EIA (enzyme immunoassay) format.

The major disadvantages mentioned above mean that a distinction cannot be made between active forms and inactive forms of MMPs, and/or that there is ambiguity in identifying the MMP detected. These limitations arise from methodological constraints on sample handling.

There is therefore still a pressing need for methods for specifically detecting the active form of a particular MMP, particularly from a complex biological sample which may contain a high concentration of proteins with a low representation of the MMP concerned.

### SUMMARY OF THE INVENTION

In the context of MMPs that exhibit strong sequence/structure homology and low substrate specificity, especially for the synthetic substrates used in tests, the present invention provides a method for specifically detecting only the active form of a particular MMP, and does so quickly and with good sensitivity in complex biological media.

The present invention relates to an in vitro method for specifically detecting in a biological sample a matrix metalloproteinase (MMP) of interest only in its active form, comprising
a) a step of placing the biological sample in contact with a ligand of the MMP of interest capable of binding to the free active site of said MMP;
b) subsequently or simultaneously with step a), a step of placing the result of step a) in contact with an antibody specific for the MMP of interest; and
c) a step of detecting the ternary complex between the MMP of interest, the ligand of the MMP of interest, and the antibody specific for the MMP of interest,

and wherein the ligand comprises a phosphinic pseudopeptide inhibitor.

Preferably, either the ligand of the MMP of interest or the antibody specific for the MMP of interest is immobilized on a solid support, and the detection is achieved either by detecting the antibody when the ligand is immobilized on the support or by detecting the ligand when the antibody is immobilized on the support.

In a first preferred embodiment, the method comprises
a) providing a solid support on which a ligand of the MMP of interest is immobilized;
b) placing the solid support in contact with the sample so as to allow attachment of the MMP of interest present in the sample to the solid support via a binding between the ligand and the MMP of interest;
c) optionally, removing the unattached MMPs and other proteins of the sample;
d) adding an antibody specific for the MMP of interest to allow formation of the ternary complex between the MMP of interest, the ligand of the MMP of interest, and the antibody specific for the MMP of interest;
e) removing the free antibodies; and
f) detecting antibodies in the ternary complex, this detection being indicative of the active form of the MMP of interest being present in the sample.
   In a second alternative embodiment, the method comprises
a) providing a solid support on which an antibody specific for the MMP of interest is immobilized;
b) placing the solid support in contact with the sample which has been previously or is simultaneously placed in contact with a ligand of the MMP of interest, so as to allow attachment of the MMP of interest present in the sample to the ligand and immobilization of the MMP of interest on the solid support via a linkage between the antibody and the MMP of interest;
c) removing the free MMPs and ligands; and
d) detecting the ligand in the ternary complex between the MMP of interest, the ligand of the MMP of interest, and the antibody specific for the MMP of interest, this detection being indicative of the active form of the MMP of interest being present in the sample.

Preferably, the method uses a solid-phase immunoassay or immunochromatographic test (ICT). The solid phase may be a membrane (flow-through), a well of a microtiter plate (EIA for example), or strips (dipstick). The ligand or antibody is detected by its covalent or non-covalent coupling to a detectable marker. The detectable marker may be selected from the group consisting of a colloidal metal, a non-metal colloid, carbon, a visible, fluorescent, luminescent or chemiluminescent tracer, a magnetic particle, a radioactive element, latex beads carrying a visible or fluorescent tracer, and an enzyme; preferably colloidal gold or an enzyme.

In one particular embodiment, the ligand is coupled to a carrier protein, preferably via a linker or spacer, in particular a polyethylene glycol linker. The carrier protein may be mono- or polyfunctionalized with the ligand of the MMP of interest. It may be serum albumin, preferably human or bovine. It may also be an enzyme such as acetylcholinesterase.

The biological sample may be a biological liquid or fluid, or a tissue or cell extract.

The MMP of interest may be selected from among MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27, and MMP-28, preferably from among MMP-2, MMP-3, MMP-8, MMP-9, MMP-10, MMP-12, MMP-13 and MMP-14, and is preferably MMP-12.

Preferably, the ligand comprises a moiety of formula (I):

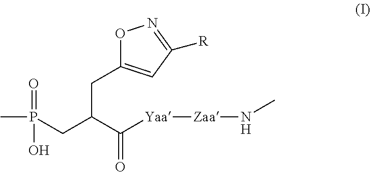

where

Yaa′ is a natural amino acid other than Asp, Pro, Gly, Cys, and Gln, in particular selected from the group consisting of Ala, Arg, Asn, Glu, His, Ile, Leu, Lys, Met, Phe, Ser, Thr, Val, Trp, and Tyr;

Zaa′ is a natural amino acid other than Pro and Cys, in particular selected from the group consisting of Ala, Arg, Asp, Asn, Gly, Gln, Glu, His, Ile, Leu, Lys, Met, Phe, Ser, Thr, Val, Trp, and Tyr;

R is selected from the group consisting of

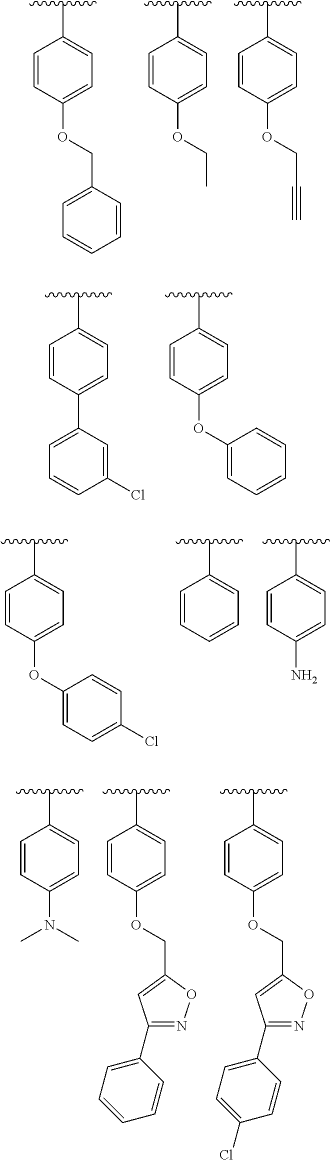

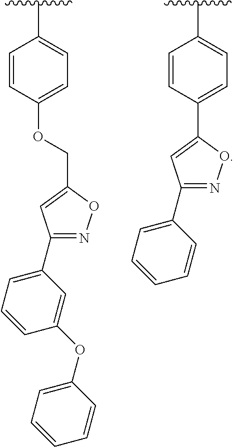 [SMILES: *c1ccc(OCc2cc(-c3cccc(Oc4ccccc4)c3)no2)cc1 ; *c1ccc(-c2cc(-c3ccccc3)no2)cc1]

In a preferred embodiment, the ligand comprises a moiety of formula (III):

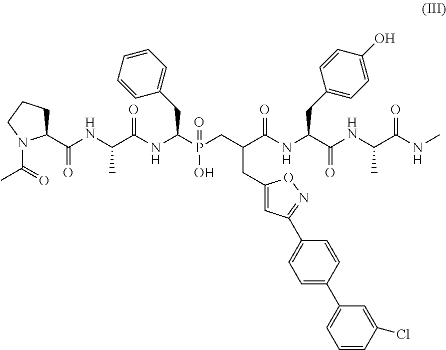 [SMILES: CNC(=O)[C@H](C)NC(=O)[C@H](Cc1ccc(O)cc1)NC(=O)C(Cc1cc(-c2ccc(-c3cccc(Cl)c3)cc2)no1)CP(=O)(O)[C@H](Cc1ccccc1)NC(=O)[C@H](C)NC(=O)[C@@H]1CCCN1C(C)=O]

The present invention also relates to a kit for specifically detecting an MMP of interest solely its active form, comprising
a) an antibody specific for the MMP of interest;
b) a ligand of the MMP of interest, comprising a phosphinic pseudopeptide inhibitor, preferably functionalizing a carrier protein;
c) optionally, a solid support on which is immobilized either the antibody or the ligand; and
d) optionally, reagents enabling detection of the antibody or ligand.

Finally, the present invention relates to the use of a method according to the invention or a kit according to the invention in a diagnostic method, in particular for the diagnosis of cancer, viral infection, osteoarthritis, rheumatoid arthritis, Dupuytren's contracture, atherosclerosis, respiratory diseases with inflammatory components such as chronic obstructive pulmonary disease, emphysema, and asthma, or neurodegenerative diseases such as multiple sclerosis, myasthenia gravis, stroke, amyotrophic lateral sclerosis, or Alzheimer's disease.

### DETAILED DESCRIPTION OF THE INVENTION

The present invention relates to a method for detecting only the active form of an MMP of interest, that is highly sensitive and easy to implement, including for complex samples. More specifically, this method combines on one hand the use of an antibody specific for the MMP of interest, thereby allowing discrimination between the MMP of interest and other MMPs present in the sample, and on the other hand the use of a ligand binding to the active site of the MMP and comprising a phosphinic pseudopeptide inhibitor, this ligand allowing discrimination between the active form of the MMP of interest and the other forms of the MMP of interest that may be present in the sample (the proform and the form inactivated by the natural inhibitor). To allow specifically detecting the active form among the proforms or inactive forms, this ligand is brought into contact with the sample, with no prior step that could modify the linkage between the MMPs and natural inhibitors or pro-peptide, such as washes, activations, or denaturation. Detection of the ternary complex between the ligand, the MMP of interest, and the specific antibody allows specific detection of the active form of the MMP of interest.

This method is novel because the previously described methods are all designed to include washing steps between attaching the MMP to the solid support and contact with the substrate, or denaturation steps. But such steps eliminate the linkage between the MMP and natural inhibitors.

Thus, the present invention relates to a method for specifically detecting in a sample a matrix metalloproteinase (MMP) of interest only in its active form, comprising
a) a step of placing the biological sample in contact with a ligand of the MMP of interest capable of binding to the free active site of the MMP;
b) subsequently or simultaneously with step a), a step of placing the result of step a) in contact with an antibody specific for the MMP of interest; and
c) a step of detecting the ternary complex between the MMP of interest, the ligand of the MMP of interest, and the antibody specific for the MMP of interest,

and wherein the ligand comprises a phosphinic pseudopeptide inhibitor.

Preferably, either the ligand of the MMP of interest or the antibody specific for the MMP of interest is immobilized on a solid support, and the detection is carried out either by detection of the antibody when the ligand is immobilized on the support or by detection of the ligand when the antibody is immobilized on the support.

The MMP of interest is preferably selected from the group consisting of MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27, and MMP-28. In one particular embodiment, it is selected from the group consisting of MMP-2, MMP-3, MMP-8, MMP-9, MMP-10, MMP-11, MMP-12, MMP-13, MMP-14 MMP-15, MMP-16, MMP-17, MMP-19, MMP-20, MMP-21, MMP-23A, MMP-23B, MMP-24, MMP-25, MMP-26, MMP-27, and MMP-28. In a preferred embodiment, it is selected from the group consisting of MMP-2, MMP-3, MMP-8, MMP-9, MMP-10, MMP-12, MMP-13, and MMP-14. In another preferred embodiment, it is selected from the group consisting of MMP-2, MMP-3, MMP-8, MMP-9, MMP-12, MMP-13, and MMP-14. In a particularly preferred embodiment, the MMP of interest is MMP-12. The MMP of interest is preferably a human MMP. However, it may be from other species in which detection of the active form has diagnostic value, such as domestic animals (dog, cat, or horse, for example), or experimental value, such as laboratory animals (mouse or rat, for example).

The test sample is preferably a biological sample. This sample may be, for example but not limited to, a sample of biological fluid or liquid, including a blood sample, lymph sample, sputum, particularly a tracheobronchial lavage, saliva, urine, bile, pancreatic juice, semen, amniotic fluid, mucus, gastric fluid, sweat, cerebrospinal fluid, synovial fluid, pleural fluid, peritoneal fluid, or pericardial fluid. This sample may also be tissue extract, in particular cell extract. Preferably, the test sample is prepared from the collected sample or from the cell or tissue extract, in particular by dilution. Dilution may be by a factor of 2, 5, 10, 20, 50, or 100. However, as MMPs are usually present in small amounts, dilution is preferably as low as possible so the sample is compatible with the method used in the test. The sample may be from a tumor. The sample may be fresh or have been frozen.

By definition, a sample is said to be complex when it is a biological sample or is prepared from such a sample. The complexity of the sample arises from the large number of different proteins present in the sample in varying concentration ratios. The concentrations of these proteins may be very high (×1000, ×10,000) compared to the biological targets of interest such as MMPs (whose concentrations can be estimated to be subnanomolar: ≦1 nM).

The first step comprises placing the biological sample in contact with a ligand of the MMP of interest.

An important point of the present method is that the biological sample is placed in the presence of the ligand of the MMP of interest with no prior step capable of changing the state of the MMP. As explained above, MMPs may be present in three states: 1) proform, meaning before cleavage of the pro-peptide, the pro-peptide being bound to the active site; 2) in active form, meaning without the pro-peptide; and 3) in inactive form, meaning without the pro-peptide but with a natural MMP inhibitor bound to the active site. The natural MMP inhibitors are TIMPs (tissue inhibitors of metalloproteinases), specifically TIMP-1, TIMP-2, TIMP-3, and TIMP-4.

The sample can, however, be subjected to dilution or extraction steps as long as this does not affect the state of the MMPs in the sample. Experimental data have shown that the method of the present invention is compatible with the media used to dilute biological samples or prepare tissue or cell extracts. However, the method does not include a step of MMP immobilization followed by washing, which is likely to change the state of the MMP before the sample is placed in contact with the ligand.

The MMP ligand is a ligand capable of binding to the free active site of the MMP of interest. It can be also considered an inhibitor, as it lastingly prevents binding of the substrate to the active site of MMP and it is not cleaved by MMP. It must be able to bind with very high affinity and to maintain this interaction during washing steps. However, it should not displace natural inhibitors bound to the MMPs. Preferably, the ligand should have an inhibition constant Ki for the MMP interest of 2 nanomolar or less, particularly a subnanomolar Ki (less than or equal to 1 nM, particularly a Ki less than 1, 0.5, or 0.1 nM). It is preferable to choose inhibitors having specificity for MMPs relative to the other proteases present in the test sample. Finally, another important parameter in the selection of inhibitors is chemical stability. Indeed, the inhibitor should not be metabolized and its structure should not be altered in the sample, meaning in biological fluids or cell extracts.

The inventors have defined the various parameters for inhibitor selection and have chosen phosphinic pseudopeptide inhibitors.

Many MMP inhibitors have been described. Some have excellent affinity for MMPs. However, these inhibitors were inappropriate despite this affinity. For example, one well-known class of inhibitors, hydroxamate inhibitors, has been the most researched in the development of MMP inhibitors. This class was not chosen, however. Indeed, they are not very specific to MMPs and interact with other metalloprotease families such as ADAM, ADAM-TS, or abundant metalloproteases such as neprilysin, which makes them difficult to use in complex samples. They are also very unstable chemically, because the hydroxamate functional group is hydrolyzed to generate a carboxylate and compounds with less affinity; this makes them difficult to use in complex media, particularly those used for preparing cell extracts.

The class of phosphinic pseudopeptides is known to those skilled in the art and, for example, has been described in Dive et al (2004, Cell Mol Life Sci, 61, 2010-2019); Fisher and Mobashery (2006, Cancer Metastasis Rev. 25, 115-136); WO00/43404, WO01/25264, WO07/062376, WO08/057254, and WO2011/023864, the description of these being hereby incorporated by reference.

Optionally, the MMP ligand can be chosen to have specificity for the MMP of interest rather than for other MMPs. However, this aspect is not important in the present invention, the important factor only being its good affinity for the MMP of interest. In a preferred embodiment of MMP12 ligands, the inhibitors are chosen from among those described in WO08/057254, WO2011/023864, or Devel et al (2006, J Biol Chem, 281, 11152-11160), the description of these being hereby incorporated by reference.

Thus, the ligand comprises or is a phosphinic pseudopeptide inhibitor.

In a preferred embodiment, the ligand comprises a moiety of formula (I):

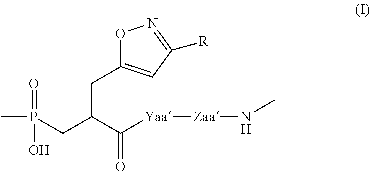

where

Yaa′ is a natural amino acid other than Asp, Pro, Gly, Cys, and Gln, in particular selected from the group consisting of Ala, Arg, Asn, Glu, His, Ile, Leu, Lys, Met, Phe, Ser, Thr, Val, Trp, and Tyr;

Zaa′ is a natural amino acid other than Pro and Cys, in particular selected from the group consisting of Ala, Arg, Asp, Asn, Gly, Gln, Glu, His, Ile, Leu, Lys, Met, Phe, Ser, Thr, Val, Trp, and Tyr;

R is selected from the group consisting of

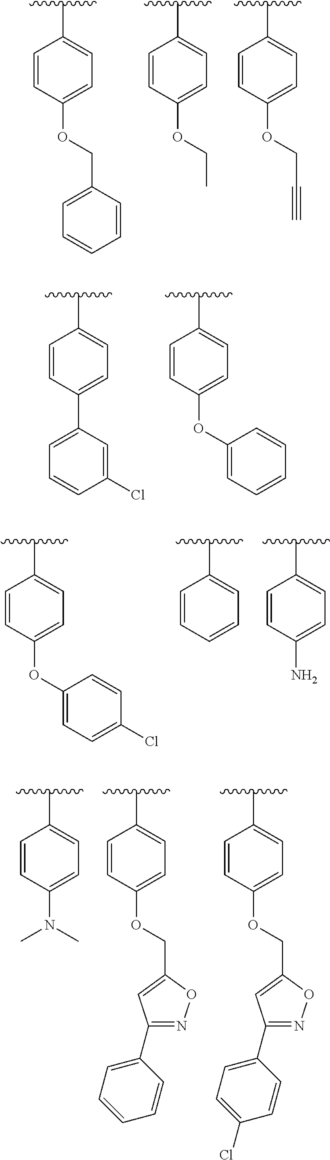

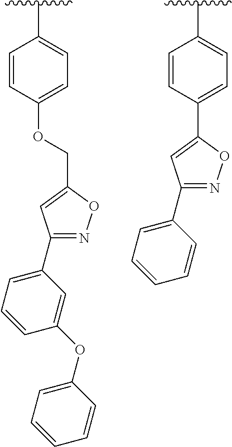 [SMILES: *c1ccc(OCc2cc(-c3cccc(Oc4ccccc4)c3)no2)cc1 ; *c1ccc(-c2cc(-c3ccccc3)no2)cc1]

The amino acids defined in the present document are represented using the three-letter symbols indicated below:

| | | |
|---|---|---|
| A | Ala | (alanine) |
| R | Arg | (arginine) |
| N | Asn | (asparagine) |
| D | Asp | (aspartic acid) |
| C | Cys | (cysteine) |
| Q | Gln | (glutamine) |
| E | Glu | (glutamic acid) |
| G | Gly | (glycine) |
| H | His | (histidine) |
| I | Ile | (isoleucine) |
| L | Leu | (leucine) |
| K | Lys | (lysine) |
| M | Met | (methionine) |
| F | Phe | (phenylalanine) |
| P | Pro | (proline) |
| S | Ser | (serine) |
| T | Thr | (threonine) |
| W | Trp | (tryptophan) |
| Y | Tyr | (tyrosine) |
| V | Val | (valine) |

More particularly, the ligand comprises a moiety of formula (II):

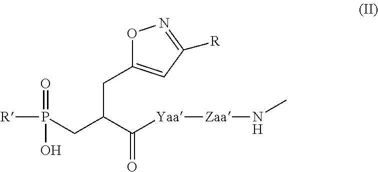

where

R, Yaa′ and Zaa′ are as defined in formula (I);

R′ is
either

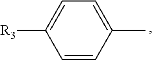

with R3 being H or Br;
or

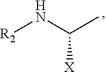

where

X is a side chain of an amino acid selected from the group consisting of Gly, Phe, Ala, Val, Leu, and Ile;

R2 is selected from the group consisting of

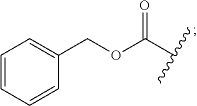 [SMILES: *C(=O)OCc1ccccc1]

—C(═O)—Waa′-Xaa′-; and —C(═O)—Vaa′-Waa′-Xaa′-Vaa′, Waa′ and Xaa′ being any natural amino acid except Cys, in particular selected from the group consisting of Ala, Arg, Asp, Asn, Gly, Gln, Glu, His, Ile, Leu, Lys, Met, Phe, Pro, Ser, Thr, Val, Trp, and Tyr.

In a very particular embodiment, the ligand is such that it comprises a moiety of formula II, where

Yaa′ is Tyr, and Zaa′ is Ala;

R is

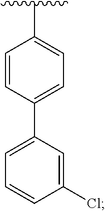 [SMILES: *c1ccc(-c2cccc(Cl)c2)cc1]

R′ is

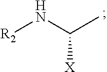

with X being the side chain of Phe and R2 being —C(═O)—Waa′-Xaa′- where Xaa′ is Ala and Waa′ is Pro.

The ligand thus comprises a moiety of formula (III):

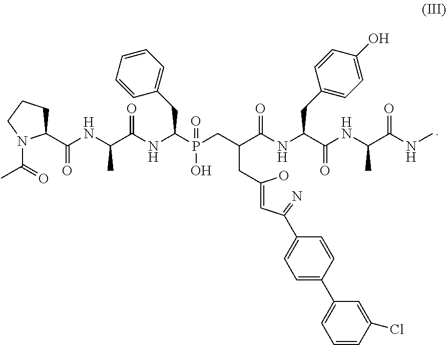 [SMILES: CNC(=O)[C@@H](C)NC(=O)[C@H](Cc1ccc(O)cc1)NC(=O)C(C/C(O)=C/C(=N)c1ccc(-c2cccc(Cl)c2)cc1)CP(=O)(O)[C@H](Cc1ccccc1)NC(=O)[C@@H](C)NC(=O)[C@@H]1CCCN1C(C)=O]

In another very particular embodiment, the ligand is such that it comprises a moiety of formula II, where

Yaa′ is Tyr, and Zaa′ is Ala;

R is

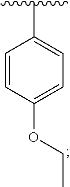 [SMILES: *c1ccc(OCC)cc1]

R′ is

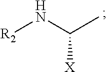

with X being the side chain of Phe and R2 being —C(═O)—Waa′-Xaa′- where Xaa′ is Ala and Waa′ is Pro.

In another very particular embodiment, the ligand is such that it comprises a moiety of formula II, where

Yaa′ is Tyr, and Zaa′ is Ala;

R is

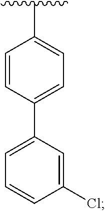 [SMILES: *c1ccc(-c2cccc(Cl)c2)cc1]

R′ is

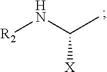

with X being the side chain of Phe and R2 being —C(═O)-Vaa′-Waa′-Xaa′- with Vaa′ being Phe, Xaa′ being Ala, and Waa′ being Pro.

Preferably, the ligand further comprises a spacer at one and/or the other end of the moiety, in particular at the Zaa′ end. Optionally, the spacer can be —CH₂—C(═O)—NH—(CH₂)₃—O—(CH₂)₂—O—(CH₂)₂—O—(CH₂)₃—NH—.

The ligand can therefore comprise a moiety of formula (III′):

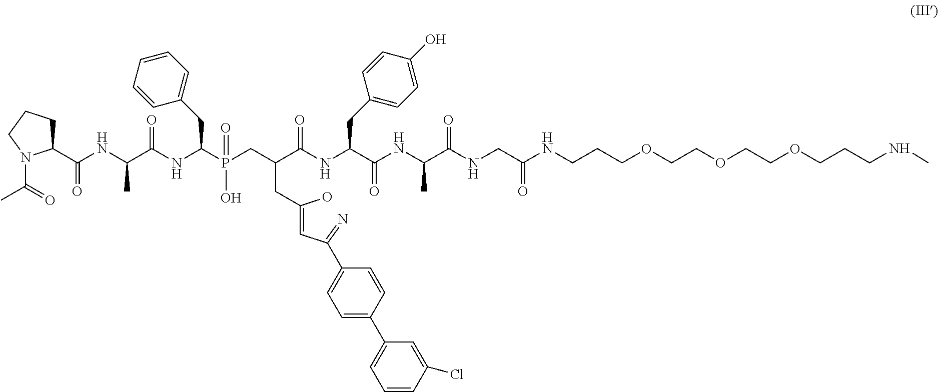 [SMILES: CNCCCOCCOCCOCCCNC(=O)CNC(=O)[C@@H](C)NC(=O)[C@H](Cc1ccc(O)cc1)NC(=O)C(C/C(O)=C/C(=N)c1ccc(-c2cccc(Cl)c2)cc1)CP(=O)(O)[C@H](Cc1ccccc1)NC(=O)[C@@H](C)NC(=O)[C@@H]1CCCN1C(C)=O]

In a preferred embodiment of the invention, the ligand is coupled to a carrier protein. The bond between carrier protein and ligand can be covalent or non-covalent.

When the bond is non-covalent, the ligand may be immobilized on the carrier protein by a pair of molecules that have affinity for one another, for example a biotin-streptavidin or a biotin-avidin couple. The ligand is thus covalently bound to biotin, preferably by a linker or spacer such as polyethylene glycol, and the carrier protein is bound to or is streptavidin. Ligand P3 is an illustration of the ligand bound to biotin.

When the bond is covalent, the carrier protein functionalized with a ligand of the MMP of interest can be any protein carrying amino acids allowing functionalization, for example lysine, histidine, tyrosine, or cysteine. As shown by the examples, the nature of this carrier protein has no impact on the detection test, because comparable results were obtained using BSA, HSA, and acetylcholinesterase as carrier protein.

The carrier protein can be monofunctionalized (carrying a single MMP ligand) or polyfunctionalized (carrying several MMP ligands). In a preferred embodiment, when the immunochromatography technique is used with immobilization of the MMP ligand on a solid support, a polyfunctionalized carrier protein is preferred.

The MMP ligand is preferably bound to the carrier protein via a linker or spacer. The primary function of this bond is to ensure the ligand is available to interact with the active site of the MMP. The bond preferably has a length of at least approximately 30 Angstrom. For example, the bond may have a length of 100 to 200 Angstrom. The complementary function, when the ligand is particularly hydrophobic, is to facilitate solubility of the ligand. One example is a polyethylene glycol (PEG) linker/spacer, particularly PEGs of at least 10 repetitions, and in particular 20-40 repetitions, for example about 30 repetitions. Other spacers are well known to those skilled in the art and can be used.

Preferably, the molecular weight of the carrier protein, or a monomer of the carrier protein when it is multimeric, is not too high, preferably less than 100 kDa.

The carrier protein must not interfere with the activity of the MMPs.

In one embodiment, the carrier protein does not exhibit enzymatic activity. Non-limiting examples of carrier proteins are serum albumin (including human or bovine), ovalbumin, tyroglobulin, tetanus toxoid or diphtheria toxoid, keyhole limpet hemocyanin (KLH), maltose binding protein (MBP), and flagellin. In a preferred embodiment, the carrier protein is serum albumin, preferably human or bovine.

In another embodiment, the carrier protein also allows detection and may have detectable enzymatic activity. It is therefore directly or indirectly conjugated to a detectable marker.

“Indirectly conjugated” is understood to mean that the protein can be attached to a detectable marker, particularly via a pair of molecules that have affinity for one another, such as a biotin-streptavidin couple. The carrier protein can thus have one or more biotins attached to its surface and the streptavidin will be conjugated to the detectable marker.

“Directly conjugated” is understood to mean that the protein has detectable markers on its surface. These detectable markers may be, for example, selected from the group consisting of a colloidal metal, a non-metal colloid, carbon, a visible, fluorescent, luminescent or chemiluminescent tracer, a magnetic particle, a radioactive element, latex beads carrying a visible or fluorescent tracer, and an enzyme whose activity can be detected. Preferably, the detectable marker is colloidal gold or an enzyme. The enzymes that can be used in this type of application are well known in the art, and we can cite acetylcholinesterase or peroxidase as illustrations.

In a particular embodiment, the carrier protein is the enzyme used as the detectable marker. Enzymes used or usable in enzyme immunoassays (EIA) or in ELISA are well known to those skilled in the art and can be used in the present invention as carrier proteins. This embodiment is of particular interest when the carrier protein functionalized with the MMP ligand is used for detection of the MMP of interest immobilized on the support in an EIA test.

Methods for coupling (or functionalization) of the MMP ligand to the carrier protein are well known to those skilled in the art. Examples include coupling by carbodiimide, glutaraldehyde, bis-diazotized benzidine, or maleimide.

The next step, which may be subsequent to or simultaneous with step a), is a step of placing the result of step a) in contact with an antibody specific for the MMP of interest.

The antibody must be able to discriminate the MMP of interest from other MMPs possibly present in the test sample. It is the specificity of the antibody that allows the method to specifically detect the MMP of interest.

The antibody specific for the MMP of interest may be polyclonal or monoclonal. It may be IgG, IgM, or IgA, preferably IgG. The antibody must link to the MMP in a manner that does not interfere with the binding of the MMP to the ligand, in other words does not bind to the active site of the catalytic domain of MMP. It can bind to the catalytic domain however, but outside of the active site thereof.

Many anti-MMP antibodies are commercially available for each MMP, for example from Sigma-Aldrich, R&D, Millipore, Aviva, GeneTex, MyBioSource.com, Genway Biotech Inc., Thermo Scientific Pierce Antibodies, Acris Antibodies GmbH, Cosmo Bio Co. Ltd., and LifeSpan BioSciences.

Finally, the method comprises a step of detecting the ternary complex between the MMP of interest, the ligand of the MMP of interest, and the antibody specific for the MMP of interest. Detection of this ternary complex can be carried out by detection of the antibody or by detection of the ligand.

In a first preferred embodiment, the ternary complex is detected by detection of the antibody. This antibody may be labeled directly or indirectly, preferably directly. For example, it may be conjugated to detectable markers, for example enzymes, gold particles, or colored, fluorescent, or radioactive markers. Alternatively, the antibody may be detected by secondary antibodies conjugated to detectable markers, such as antibodies against the antibody specific for the MMP. For example, if the antibody specific to the MMP is a mouse immunoglobulin, the secondary antibody is specific for mouse immunoglobulins. In addition, the antibody may be conjugated to a member of a pair of molecules that have affinity for one another, for example conjugated to biotin. The other member, in particular streptavidin, is conjugated to the detectable marker.

In a first alternative embodiment, the ternary complex is detected by detection of the ligand. This can be done directly if the ligand is conjugated to a detectable marker or if the carrier protein is an enzyme having detectable activity. It can also be done indirectly if the ligand or the carrier protein is conjugated to a member of a pair of molecules that have affinity for one another, for example conjugated to biotin. The other member, in particular streptavidin, is conjugated to the detectable marker. A final option for the labeling is to use antibodies directed against the carrier protein.

The method preferably comprises immobilization of the ternary complex on a solid support. The solid support may be a surface, a bead, or a tube. The solid support may be any support that is compatible with an immunoassay. Many materials can be used as the support, such as nitrocellulose, nylon, cellulose acetate, glass fibers, or other porous polymers.

Immobilization is achieved either via the ligand or via the antibody. When the MMP is immobilized by means of the ligand, then detection of the ternary complex occurs by detection of the antibody. Conversely, when the MMP is immobilized by means of the antibody, then detection of the ternary complex occurs by detection of the ligand.

Thus, in a first particularly preferred embodiment where immobilization of the ternary complex is achieved via the ligand, the method comprises
a) providing a solid support on which a ligand of the MMP of interest is immobilized;
b) placing the solid support in contact with the sample so as to allow attachment of the MMP of interest present in the sample to the solid support via a binding between the ligand and the MMP of interest;
c) optionally, removing the unattached MMPs and other proteins of the sample;
d) adding an antibody specific for the MMP of interest to allow formation of the ternary complex between the MMP of interest, the ligand of the MMP of interest, and the antibody specific for the MMP of interest;
e) removing the free antibodies; and
f) detecting antibodies in the ternary complex, their detection being indicative of the active form of the MMP of interest being present in the sample.

Steps c) and e) may be achieved by performing washing steps.

Optionally, steps b) and d) can be reversed. Thus, the sample is placed in contact with the antibody, then this mixture is placed in contact with the solid support. Alternatively, steps b) and d) can be simultaneous Thus, the sample and the antibody are placed in contact with the solid support.

In this embodiment the ligand may be immobilized directly on the solid support, optionally via a linker/spacer. Preferably, the ligand is bound to a carrier protein which is immobilized on the solid support. The carrier protein may be mono- or polyfunctionalized with the ligand. In one particular embodiment, the carrier protein is serum albumin, in particular human or bovine.

The present invention thus concerns a solid support on which a ligand as defined above has been immobilized. Preferably, the ligand is bound to a carrier protein immobilized on the solid support. The carrier protein may be mono- or polyfunctionalized with the ligand. In one particular embodiment, the carrier protein is serum albumin, in particular human or bovine. The solid support may be a strip (suitable for immunochromatography), a well of a microtiter plate, a filter or membrane, or a dipstick strip.

The present invention relates in particular to two types of test.

The first is an immunochromatographic test. This method is well known to the skilled person. The support is preferably a strip suitable for chromatography. The ligand is immobilized on the test area. In a preferred embodiment, a carrier protein polyfunctionalized with the ligand (carrying several ligands) is immobilized. Alternatively, a carrier protein monofunctionalized with the ligand (carrying one ligand) can also be immobilized. Preferably, the support further comprises a control area, this area having a means for attaching anti-MMP antibodies, for example antibodies against the anti-MMP antibodies. For example, if the anti-MMP antibody is a mouse antibody, an antibody specific for mouse antibodies will be used. The same applies for rabbit, rat, or goat antibodies. The sample is placed in contact with the ligand by depositing the sample on the side of the sample deposit area, then migration of the sample occurs. Optionally, one or more washes are then performed. The active MMP initially having the free active site is immobilized on the test area by interaction with the ligand. The other forms are removed from the test area because they are not attached to that area. Provided experimental data show that this system allows selective immobilization of the active forms of the MMP, as the proform and the form inactivated by inhibitors are not retained and therefore are not detected; and this system does so in various environments and with complex samples. The MMP immobilized on the test area is then detected by adding an antibody specific for the MMP of interest. This antibody may be labeled directly or indirectly, preferably directly. This embodiment has proven to be specific for the active form, sensitive (about 1.2 ng/ml in buffer), and compatible with complex media (sensitivity of 10-20 ng/ml in complex samples).

Alternatively, the sample can be placed on the strip at the deposit area. It migrates along the strip and is brought together with an area having anti-MMP antibody capable of migrating (not immobilized on the solid support). The sample and the antibody then migrate along the strip to the test and control areas.

It is also possible to detect different MMPs of interest. Thus, the ligand is chosen to be compatible with (in other words able to bind to) the MMPs of interest, and each strip is used with an antibody specific for a different MMP of interest. For example, if two MMPs are to be detected, there will be one strip used with an antibody specific for the first MMP and another strip used with an antibody specific for the second MMP.

The invention also relates to a kit for conducting the test. This kit comprises:
a strip comprising a test area on which the ligand of the MMP of interest is immobilized. Preferably, the ligand is coupled to a carrier protein, in particular via a linker or spacer. The carrier protein is preferably polyfunctionalized with the ligand. Preferably, the strip further comprises a control area on which is immobilized a means for attaching the anti-MMP antibodies. The strip further comprises a sample deposit area, and optionally an area supporting anti-MMP antibodies downstream thereof and before the test areas.
an antibody specific for the MMP of interest, preferably labeled with a colored particle (for example a gold or latex particle), a fluorophore, or a radioisotope; and
optionally, reagents such as wash buffers, dilution or extraction buffers.

When several different MMPs of interest are detected, the kit contains at least one strip per MMP of interest and further comprises one specific antibody per MMP of interest. For example, if two MMPs are to be detected, there will be at least one antibody specific for the first MMP and another specific for the second. The kit may contain a single type of strips and as many specific antibodies as there are MMPs of interest.

The second is a solid phase immunoassay, preferably an enzyme immunoassay. The solid support may be a membrane or a filter (flow-through), a well of a microtiter plate (for example EIA), or strips (dipstick). The ligand is immobilized on the solid support. In a preferred embodiment, a carrier protein monofunctionalized with the ligand (carrying a ligand) is immobilized. Alternatively, a carrier protein polyfunctionalized with the ligand (carrying several ligands) may also be immobilized. The sample is then placed in contact with the immobilized ligand. After incubation, one or more washes are possibly performed. Experimental data which are provided show that this system allows selective immobilization of the active forms of the MMP, as the proform and the form inactivated by inhibitors are not retained and therefore not detected; and this system does so in various environments and with complex samples. The antibody specific for the MMP of interest is then added, and one or more washes are optionally performed prior to antibody detection. The antibody is then detected.

When the solid support is a membrane or filter (flow-through), the ligand is immobilized on the membrane or filter, preferably as well as the control means (antibody attachment means), as separate sites. Next, the sample is applied to the membrane or the filter and passes through the membrane or filter in particular by capillarity. The antibody is then applied to reveal the presence of the ligand-MMP complex. Washing steps may also be added before or after addition of the antibody.

When the solid support is a well of a microtiter plate, an enzyme immunoassay is preferred for antibody detection. This antibody may be directly coupled to a detectable marker, preferably an enzyme, or may be indirectly coupled thereto via a biotin-streptavidin pair or a secondary antibody coupled to the marker. These techniques are well known to those skilled in the art. The enzyme may, for example, be a peroxidase or an acetylcholinesterase. This embodiment has been shown to be specific for the active form, sensitive (about 20 pg/ml in buffer), and compatible with complex media (sensitivity of 200 pg/ml in complex samples).

Finally, when the solid support is a dipstick, the solid support is preferably a non-porous support. The ligand is immobilized on the support at a separate site. Preferably, a control means (antibody attachment means) is also immobilized at a separate site. The dipstick is dipped into the sample, and then into a solution of antibody specific for the MMP to be detected. Preferably, the dipstick is dipped into washing solutions between these steps. Lastly, the presence of the antibody is detected.

In this embodiment, it is also possible to detect a plurality of different MMPs of interest. Thus, the ligand is chosen to be compatible with (in other words able to bind to) the MMPs of interest, and several antibodies specific for each of the different MMPs of interest are used. For example, if two MMPs are to be detected, there will be one solid support used with an antibody specific for the first MMP and another used with an antibody specific for the second. If the solid support is a microtiter plate well, two separate wells are used. If the solid support is a membrane, filter, or strip, two solid supports of the same type will be used.

The invention also relates to a kit for conducting the test. The kit comprises:
a solid support on which the ligand of the MMP of interest has been immobilized. Preferably, the ligand is coupled to a carrier protein, in particular via a linker or spacer. Optionally, it further comprises immobilization of an antibody attachment means on a site separate from the site where the ligand is immobilized, in order to conduct a control.
an antibody specific for the MMP of interest. The antibody may be directly coupled to a detectable marker, for example an enzyme. Alternatively, it may be coupled to a molecule allowing non-covalent bonding to the detectable marker, for example biotin; and
optionally, reagents such as a detectable marker, for example the enzyme, coupled to a molecule allowing linkage to the antibody such as a secondary antibody or streptavidin, the substrate forming a colored or fluorescent product after action of the enzyme, and/or wash buffers, dilution or extraction buffers.

When several different MMPs of interest are detected, the kit comprises one specific antibody per MMP of interest. For example, if two MMPs are to be detected, the kit comprises an antibody specific for the first MMP and an antibody specific for the second.

In one particular second alternative embodiment in which immobilization of the ternary complex occurs via the antibody, the method comprises
a) providing a solid support on which an antibody specific for the MMP of interest is immobilized;
b) placing the solid support in contact with the sample which has been previously or is simultaneously placed in contact with a ligand of the MMP of interest, so as to allow binding of the MMP of interest present in the sample to the ligand and immobilization of the MMP of interest on the solid support via a linkage between the antibody and the MMP of interest;
c) optionally, removing the ligands and free MMPs and other proteins of the sample; and
d) detecting the ligand in the ternary complex between the MMP of interest, the ligand of the MMP of interest, and the antibody specific for the MMP of interest, this detection being indicative of the presence in the sample of the active form of the MMP of interest.

In this embodiment, as explained above, it is crucial that the sample be brought into contact with the ligand prior to or simultaneously with any step of immobilizing the MMP on the solid support and washing.

The present invention therefore also relates to a ligand of MMPs as described above coupled to a detectable marker. Preferably, it relates to a carrier protein functionalized with the ligand. In one embodiment, the carrier protein is an enzyme whose activity is detectable. Alternatively, the carrier protein is coupled to a detectable marker. In one particular embodiment, the carrier protein is serum albumin, in particular human or bovine.

The present invention relates in particular to two types of test.

The first is an immunochromatographic test. In this embodiment, the antibody specific for the MMP of interest is immobilized on the test area. The sample is incubated with the ligand. Preferably, the ligand is bound to a carrier protein. The carrier protein may be mono- or polyfunctionalized with the ligand. Next, the sample brought into contact with with ligand is deposited on the side of the sample deposit area and migration occurs. Optionally, one or more washes are then conducted.

Alternatively, the sample can be deposited on the strip at a deposit area. It is brought into contact with the ligand after the sample migrates to an area containing the ligand, downstream but before the test area. The two together then migrate to the test area and come into contact with anti-MMP antibodies immobilized on the solid support.

The antibody-MMP-ligand ternary complex is detected via detection of the ligand. Indeed, in this configuration, all forms of the MMP of interest are retained in the test area. However, only its active forms thereof are detected because detection is via the ligand which only binds to the active form, which is the only form with an active site available. To allow detection of the ligand, it is directly or indirectly coupled to a detectable marker. When using a carrier protein functionalized with the ligand, it is also possible to use a labeled antibody specific for the carrier protein for detection.

Preferably, the solid support also comprises a control area able to bind the ligand. For example, an MMP capable of binding the ligand may be immobilized on the solid support.

In this embodiment, it is also possible to detect several different MMPs of interest. Thus, the ligand is chosen to be compatible with (in other words able to bind to) the MMPs of interest. Each strip can be used with an antibody specific for one of the different MMPs of interest immobilized on the test area. For example, if two MMPs are to be detected, there will be one strip having a test area on which is immobilized an antibody specific for the first MMP, and another having a test area on which is immobilized an antibody specific for the second. Alternatively, the strip comprises separate test areas, on which an antibody having a different specificity is immobilized. Thus, if two MMPs are to be detected, there will be one strip with two test areas, the first where an antibody specific for the first MMP is immobilized, and the other providing a test area where an antibody specific for the second is immobilized.

The invention also relates to a kit for conducting the test. This kit comprises:
a strip comprising a test area where an antibody specific for the MMPs of interest is immobilized, and preferably a control area where a ligand attachment means, for example an MMP, has been immobilized;
the ligand of the MMP of interest, preferably labeled with a colored particle (for example a gold or latex particle), a fluorophore, or a radioisotope. Preferably, the ligand is coupled to a carrier protein, in particular via a linker or spacer; and
optionally, reagents such as wash buffers, dilution or extraction buffers.

When several different MMPs of interest are to be detected, the kit may contain one strip per MMP of interest on which an antibody specific for one of the MMPs of interest has been immobilized. For example, if two MMPs are to be detected, the kit comprises a strip having a test area where an antibody specific for the first MMP is immobilized, and another having a test area where an antibody specific for the second is immobilized. Alternatively, the kit may contain a strip comprising a plurality of test areas on which an antibody specific for one of the MMPs of interest has been immobilized. For example, if two MMPs are to be detected, the kit comprises a strip having two test areas, the first where an antibody specific for the first MMP is immobilized, and another test area where an antibody specific for the second is immobilized.

The second is a solid phase immunoassay, preferably an enzyme immunoassay. The solid phase may be a membrane or a filter (flow-through), a well of a microtiter plate (for example EIA), or strips (dipstick). The antibody is immobilized on the solid support. Either the sample is pre-incubated with the ligand prior to placing it in contact with the antibody immobilized on the solid support, or the sample is placed in contact with the antibody immobilized on the solid support simultaneously with the ligand. In a preferred embodiment, the sample is pre-incubated with the ligand prior to contact with the immobilized antibody. Preferably, the ligand is bound to a carrier protein. The carrier protein may be mono- or polyfunctionalized with the ligand. Next, one or more washes can be performed. Lastly, the presence of the ligand on the solid support is detected. As before, all forms of the MMP of interest are retained on the solid support, and only its active forms are detected because the detection is done via the ligand which binds only to the active form. In one particular embodiment, the functionalized carrier protein is a detectable marker, for example an enzyme capable of generating a colored or fluorescent signal upon hydrolysis of its substrate. In another embodiment, the ligand is coupled, directly or indirectly via the carrier protein, to a member of a pair of molecules having an affinity for each other, in particular a member of a biotin-streptavidin or biotin-avidin pair, in particular biotin. The other member of the pair is bound to a detectable marker.

When the solid support is a membrane or filter (flow-through), the antibody is immobilized on the membrane or filter, preferably as well as the control means (ligand attachment means such as an MMP), as separate sites. The sample, previously brought into contact with the ligand, is then applied to the membrane or filter and passes through the membrane or filter, in particular by capillarity. The ligand immobilized on the membrane or filter is then detected. Washing steps may also be added before or after addition of the sample.

When the solid support is a well of a microtiter plate, an enzyme immunoassay is preferred for ligand detection. The ligand, or the carrier protein, may be directly coupled to a marker, preferably an enzyme, or may be indirectly coupled thereto via a biotin-streptavidin pair or a secondary antibody coupled to the detectable marker. These techniques are well known to those skilled in the art. The enzyme may, for example, be a peroxidase or an acetylcholinesterase.

Finally, when the solid support is a dipstick, the solid support is preferably a non-porous support. The antibody is immobilized on the support at a separate site. Preferably, a control means (ligand attachment means such as an MMP) is also immobilized at a separate site. The dipstick is then immersed in the sample in the presence of the ligand or pre-incubated with it beforehand, then the ligand immobilized on the support is detected. Preferably, between these steps, the dipstick is soaked in wash solutions.

In this embodiment, it is also possible to detect a plurality of different MMPs of interest. In this case, the ligand is chosen to be compatible with (in other words able to bind to) the MMPs of interest, and a set of wells or separate test areas have an immobilized antibody specific for one of the various MMPs of interest to be detected. For example, if two MMPs are to be detected, there will be one test area with an antibody specific for the first MMP and another with an antibody specific for the second. Alternatively, separate solid supports can be used for each of the MMPs to be detected.

The invention also relates to a kit that allows to conduct the test. This kit comprises:
a solid support comprising a test area where the antibody specific for the MMP of interest has been immobilized. Preferably, the solid support further comprises a control area where a ligand attachment means, for example an MMP, has been immobilized.
a ligand of the MMP of interest. Preferably, the ligand is coupled to a carrier protein, in particular via a linker or spacer. The carrier protein may be a detectable marker, preferably an enzyme. Alternatively, the ligand may be coupled to a molecule enabling a non-covalent bond to the marker, for example a biotin; and
optionally, reagents such as the enzyme coupled to a molecule allowing linkage to the ligand such as an antibody specific for the carrier protein or streptavidin, the substrate forming a colored or fluorescent product after action of the enzyme, and/or wash buffers, dilution or extraction buffers.

When several different MMPs of interest are detected, the kit comprises a set of test areas each having an immobilized antibody specific for one of the different MMPs of interest to be detected. For example, if two MMPs are to be detected, there will be a test area with an antibody specific for the first MMP and another with an antibody specific for the second.

In one particularly preferred embodiment of the methods and kits, the MMP of interest is MMP12, preferably human MMP12.

In some particular embodiments, the method further allows quantifying the active form of a specific MMP.

Since deregulation of the activity of MMPs constitutes a significant part of the pathogenic mechanisms associated with many diseases, the methods and kits of the present invention can be used for diagnosis. Such diseases include, for example, the destruction of cartilage and bone in rheumatoid arthritis and osteoarthritis, tissue remodeling during invasive tumor growth or tumor angiogenesis, degradation of myelin basic protein in neuroinflammatory diseases, blood-brain barrier integrity loss after a brain injury, increased matrix turnover in stenotic lesions, loss of tone of the aortic wall in aneurysms, tissue degradation in gastric ulceration, liver fibrosis, weakened connective tissues in periodontal disease, acute lung injury, and acute respiratory distress syndrome.

The present invention therefore relates to the use of a method according to the invention or of a kit according to the invention in a diagnostic method, in particular for the diagnosis of cancer, viral infection, osteoarthritis, rheumatoid arthritis, Dupuytren's contracture, atherosclerosis, respiratory diseases with inflammatory components such as chronic obstructive pulmonary disease, emphysema, and asthma, or neurodegenerative diseases such as multiple sclerosis, myasthenia gravis, stroke, amyotrophic lateral sclerosis, or Alzheimer's disease.

For example, MMP-1 will be used for amyotrophic lateral sclerosis; MMP-2 for cancer, Dupuytren's contracture, myasthenia gravis, or amyotrophic lateral sclerosis; MMP-3 for multiple sclerosis, stroke, or Alzheimer's disease; MMP-8 for asthma or multiple sclerosis; MMP-9 for cancer, multiple sclerosis, stroke, or Alzheimer's disease; MMP-10 for Alzheimer's disease; MMP-12 for atherosclerosis or respiratory diseases with inflammatory components such as COPD; MMP-13 for osteoarthritis or rheumatoid arthritis; and MMP-14 for Dupuytren's contracture.

Other features and advantages of the present invention will become apparent on reading the following examples, which are to be considered as illustrative and not limiting.

### DESCRIPTION OF FIGURES

FIG. 1: Detection of MMP12h on strips coupled with the use of OR-mAb12h tracer. BSA strip polyfunctionalized with P1 in the presence (1) or absence (2) of MMP12h, (3) BSA strip non-functionalized with P1 in the presence of MMP12h, (4) BSA strip polyfunctionalized with P1 in the presence of a proform of MMP12h, (5) and (6) BSA strip polyfunctionalized with P1 in presence of MMP12h previously incubated with a competitive inhibitor of MMP12h at a concentration of 100 nM (°) or 1 μM (°°).

FIG. 2: Detection of MMP12h on BSA strips polyfunctionalized with P1 paired with the use of OR-mAb12h tracer, with varying concentration of MMP12h in the sample, as well as with two buffers tested: (7)-(11): buffer of 50 mM Tris-HCl pH 6.8, 10 mM CaCl₂, 0.01% Brij; (12)-(14): 50/50 mixture of 50 mM Tris-HCl pH 6.8, 10 mM CaCl₂, 0.01% Brij buffer and of 100 mM Tris-HCl pH 7.4, 0.5% Tween 20, 1% Chaps, 0.1% NaN₃ buffer.

FIG. 3: Detection of MMP12h on BSA strips polyfunctionalized with P1 paired with the use of OR-mAb12h tracer, with varying type of MMP in the sample.

FIG. 4: Detection of MMP12h in mixed MMPs on BSA strips polyfunctionalized with P1 paired with the use of OR-mAb12h tracer.

FIG. 5: Detection of MMP12h in homogenates of cytosolic proteins on BSA strips polyfunctionalized with P1 paired with the use of OR-mAb12h tracer.

FIG. 6: Detection of MMP12h in bronchoalveolar lavages (BAL) on BSA strips polyfunctionalized with P1 paired with the use of OR-mAb12h tracer.

FIG. 7: Detection of MMP13h on BSA strips polyfunctionalized with P1 paired with the use of OR-mAb13h tracer.

FIG. 8: Detection of MMP12m on BSA strips polyfunctionalized with P1 paired with the use of OR-polyAb12m tracer, in buffered medium or in a protein mixture.

FIG. 9: Detection of MMP12h on BSA-P1 (42), HSA-P2 (43), or mAb12h (44)-(48) strips in the presence (42)-(44), (46) and (48), or absence (45) and (47) of MMP12h.

FIG. 10: Detection of MMP12h by EIA on BSA-P1 plate with increasing concentrations of MMP12h, either in a buffer with or without preincubation of the MMP12h with 1 μM competitive inhibitor of MMP, or in the presence of a mixture of cytosolic proteins.

FIG. 11: Detection of MMP12h by EIA on BSA-P1 plate with testing of three buffered media in the presence or absence of 1 μM competitive inhibitor of MMP.

FIG. 12: Detection of MMP12h by EIA on BSA-P1, P2-HSA, or mAb12h plate in the presence of increasing concentrations of MMP12h from a mixture of cytosolic proteins, with or without preincubation of the sample with 1 μM competitive inhibitor of MMP.

FIG. 13: Detection of MMP12h by EAI on mAb12h plate in the presence of buffer, of active MMP12h, or of inactive MMP12h, and on the left: with preincubation of the sample in the well before addition of biot-HSA-P2; or on the right: with simultaneous incubation of the sample and biot-HSA-P2 in the well.

FIG. 14: Detection of MMP12h by EIA on HSA-P2 or mAb12h plate in the presence of MMP12h or ProMMP12h. For mAb12h plates, Biot-P2-HSA tracer was added either after attaching MMP12h on the plate or to the sample before attaching MMP12h on the plate.

FIG. 15: Detection of MMP12h by EIA on HSA-plate P2 in bronchoalveolar lavages (BAL) containing increasing concentrations of MMP12h, with or without preincubation of the sample with a competitive inhibitor of MMP.

FIG. 16: Detection of MMP12h by EIA on polyAb12h plate in the presence of increasing concentrations of MMP12h, in a sample with a mixture of cytosolic proteins or with a reference buffer, the tracer being ligand P3 which comprises biotin.

FIG. 17: Detection of MMP12h by EIA on mAb12h plate in the presence of increasing concentrations of MMP12h, in a sample with a mixture of cytosolic proteins or with a reference buffer, the tracer being ligand P3 which comprises biotin.

FIG. 18: Detection of MMP12h by EIO on mAb12h plate in the presence of increasing concentrations of MMP12h, in a sample with different buffers, the tracer being ligand P3 which comprises biotin.

FIG. 19: Quantitation, using a fluorogenic substrate, of human MMP-2, MMP-9, MMP-12, MMP-13, and MMP-14 present in supernatants following incubation of samples on BSA-P1 and HSA-P2 plates.

FIG. 20: Detection of MMP12h by EIA on CAS plate in the presence of increasing concentrations of MMP12h, of tracer AchE(G4)-P1, and of monoclonal antibody mAb12h: absorbances measured at 1 hour as a function of the concentration of active MMP12h.

FIG. 21: Detection of MMP12h by EIA on mAb12h plate in the presence of buffer, active MMP12h, or inactive MMP12h, and on the left: in the presence of AChE(G4)-P1; or on the right: in the presence of biot-HSA-P2 in the well.

FIG. 22: Detection of MMP2h by EIA on BSA-P1 plate with increasing concentrations of MMP2h in a buffer.

FIG. 23: Detection of MMP9h by EIA on BSA-P1 plate with increasing concentrations of MMP9h in a buffer.

### EXAMPLES

### Materials and Methods

The format of the various strips used in the experiments was:
BSA-P1 strip: strip on which BSA polyfunctionalized with P1 is absorbed.
HSA-P2 strip: strip on which HSA monofunctionalized with P2 is absorbed.
mAb12h strip: strip on which anti-human MMP12 antibody is absorbed.

The format of the various plates used in the experiments was:
BSA-P1 plate: plate on which BSA polyfunctionalized with P1 is absorbed.
HSA-P2 plate: plate on which HSA monofunctionalized with P2 is absorbed.
mAb12h plate: plate on which anti-human MMP12 antibody is absorbed.
polyAb12 plate: plate on which anti-human or murine MMP12 antibody is absorbed.

The tracers used are the following:
OR-mAb12h: anti-human MMP12 antibody coupled to a gold bead, used with BSA-P1 and BSA-P2 strips.
OR-BSA-P1: BSA polyfunctionalized with P1 coupled to a gold bead, used with a mAb12h strip.
OR-BSA-P2: BSA monofunctionalized with P2 coupled to a gold bead, used with a mAb12h strip.
OR-HSA-P2: HSA monofunctionalized with P2 coupled to a gold bead, used with a mAb12h strip.
Biotin-HSA-P2 or biot-HSA-P2: HSA monofunctionalized with P2 coupled to biotin.
Biotin-BSA-P2 or P2-biot-BSA: BSA monofunctionalized with P2 coupled to biotin.
AChE(G4)-P1: AChE(G4) polyfunctionalized with P1

Ligands P1, P2 and P3

Structures of Ligands P1, P2 and P3

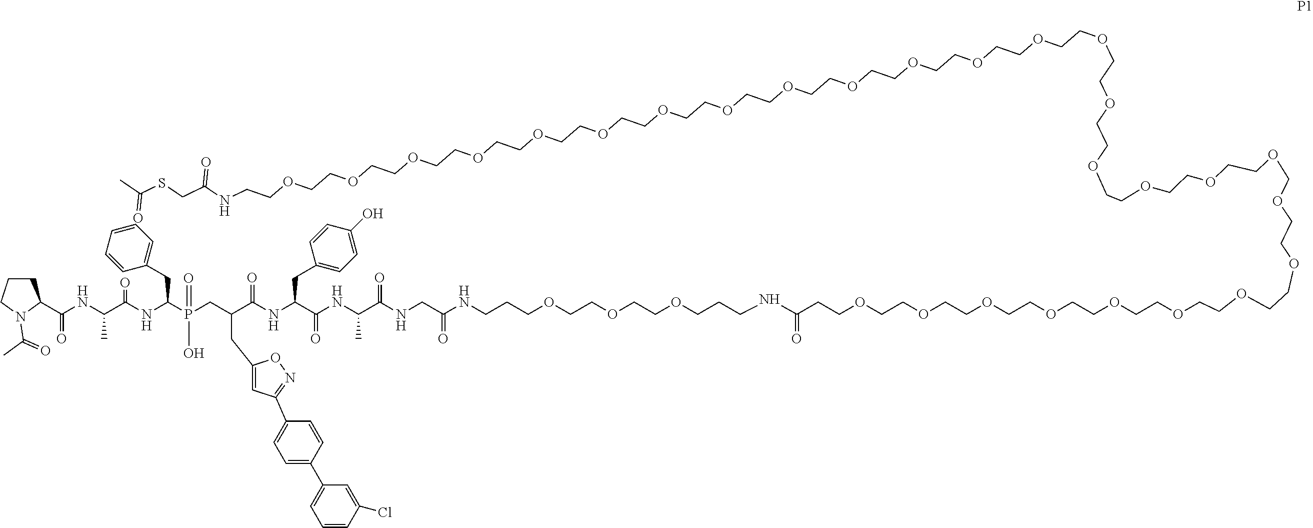 [SMILES: "P1" ; CC(=O)SCC(=O)NCCOCCOCCOCCOCCOCCOCCOCCOCCOCCOCCOCCOCCOCCOCCOCCOCCOCCOCCOCOCCOCCOCCOCCOCCOCCOCCOCCOCCC(=O)NCCCOCCOCCOCCCNC(=O)CNC(=O)[C@H](C)NC(=O)[C@H](Cc1ccc(O)cc1)NC(=O)C(Cc1cc(-c2ccc(-c3cccc(Cl)c3)cc2)no1)CP(=O)(O)[C@H](Cc1ccccc1)NC(=O)[C@H](C)NC(=O)[C@@H]1CCCN1C(C)=O]

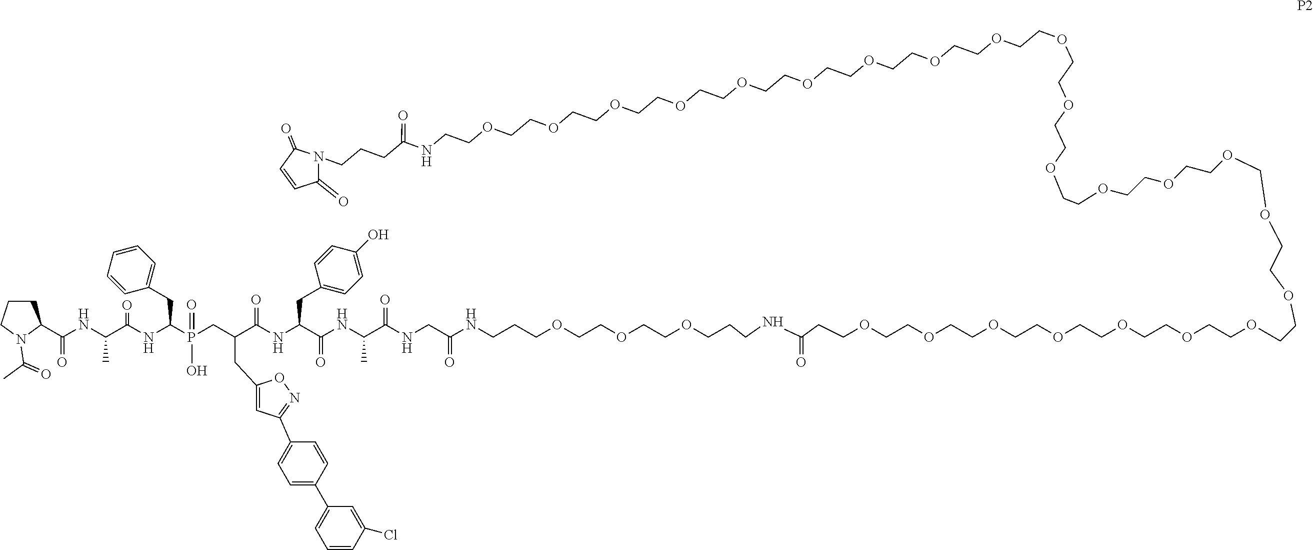 [SMILES: "P2" ; CC(=O)N1CCC[C@H]1C(=O)N[C@@H](C)C(=O)N[C@@H](Cc1ccccc1)P(=O)(O)CC(Cc1cc(-c2ccc(-c3cccc(Cl)c3)cc2)no1)C(=O)N[C@@H](Cc1ccc(O)cc1)C(=O)N[C@@H](C)C(=O)NCC(=O)NCCCOCCOCCOCCCNC(=O)CCOCCOCCOCCOCCOCCOCCOCCOCCOCOCCOCCOCCOCCOCCOCCOCCOCCOCCOCCOCCOCCOCCOCCOCCNC(=O)CCCN1C(=O)C=CC1=O]

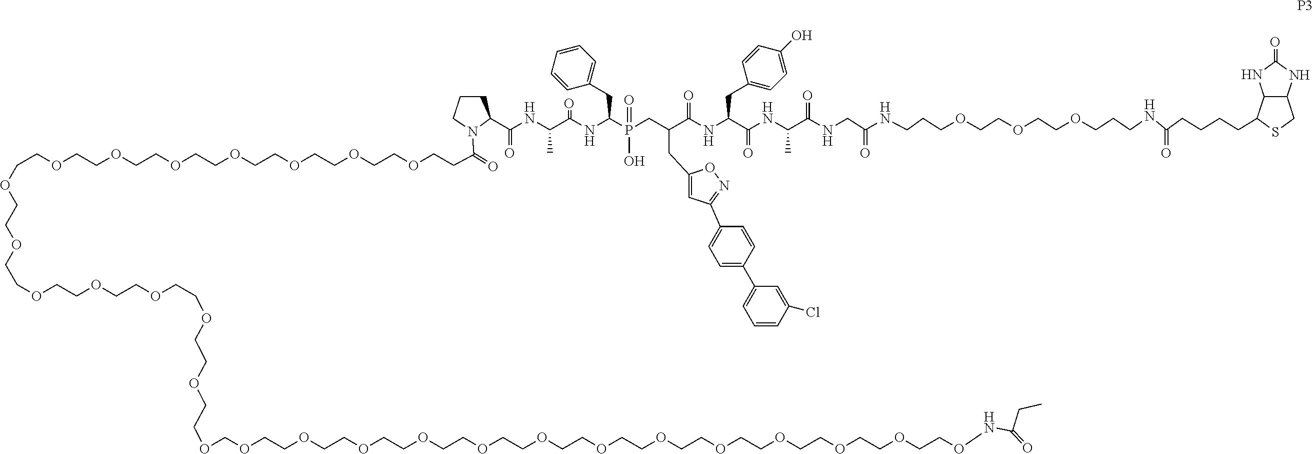 [SMILES: "P3" ; CCC(=O)NOCCOCCOCCOCCOCCOCCOCCOCCOCCOCCOCCOCCOCOCCOCCOCCOCCOCCOCCOCCOCCOCCOCCOCCOCCOCCOCCOCCC(=O)N1CCC[C@H]1C(=O)N[C@@H](C)C(=O)N[C@@H](Cc1ccccc1)P(=O)(O)CC(Cc1cc(-c2ccc(-c3cccc(Cl)c3)cc2)no1)C(=O)N[C@@H](Cc1ccc(O)cc1)C(=O)N[C@@H](C)C(=O)NCC(=O)NCCCOCCOCCOCCCNC(=O)CCCCC1SCC2NC(=O)NC21]

Synthesis Strategy for Ligand P1

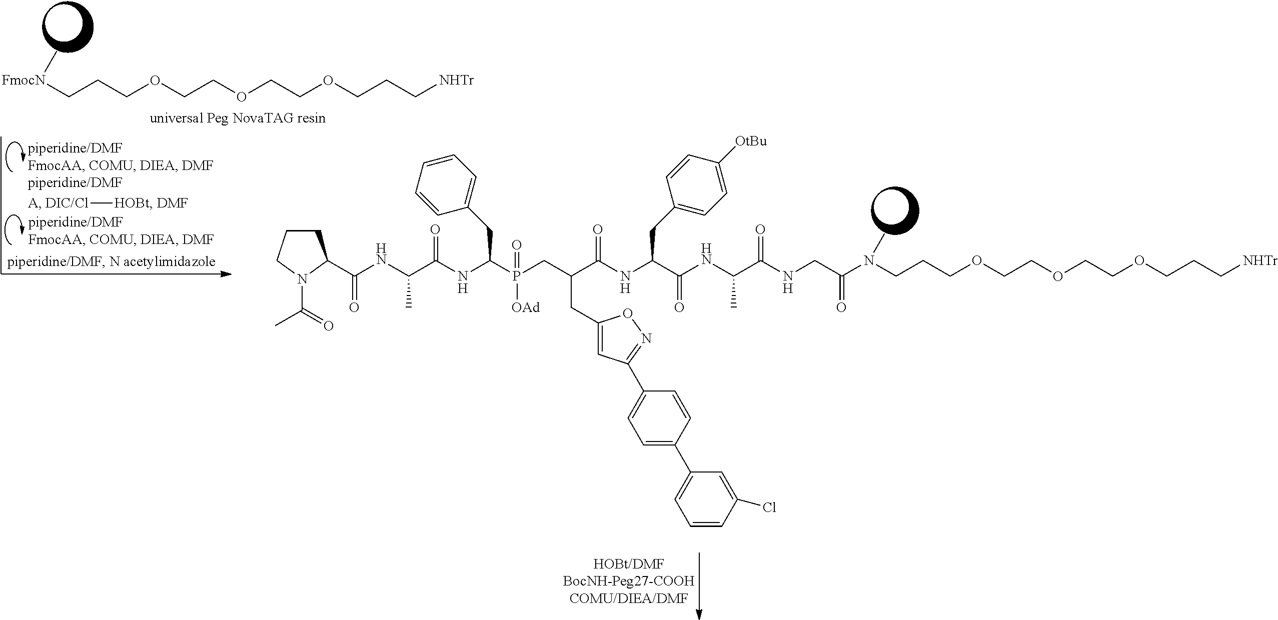

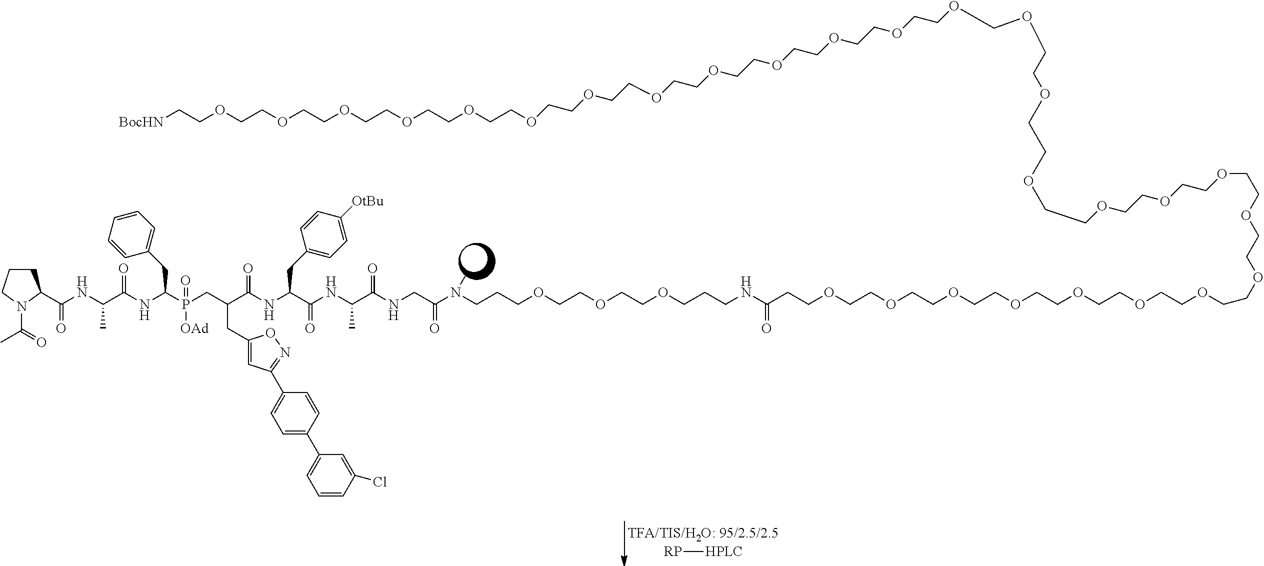

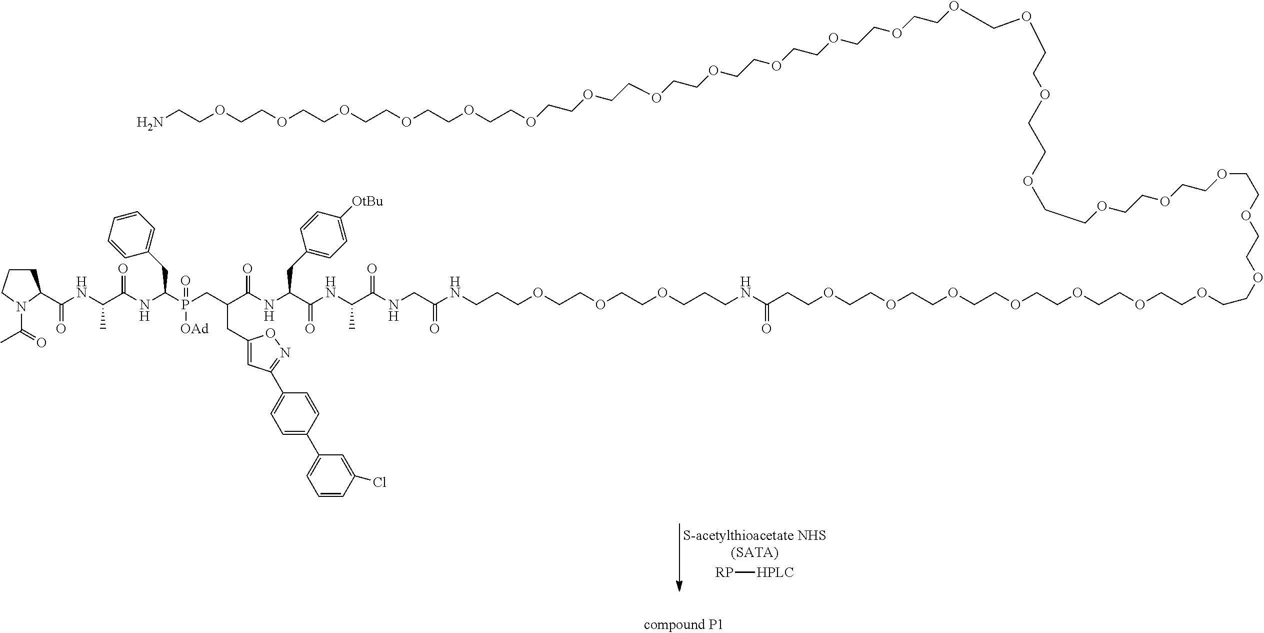

Synthesis Strategy for Ligand P2

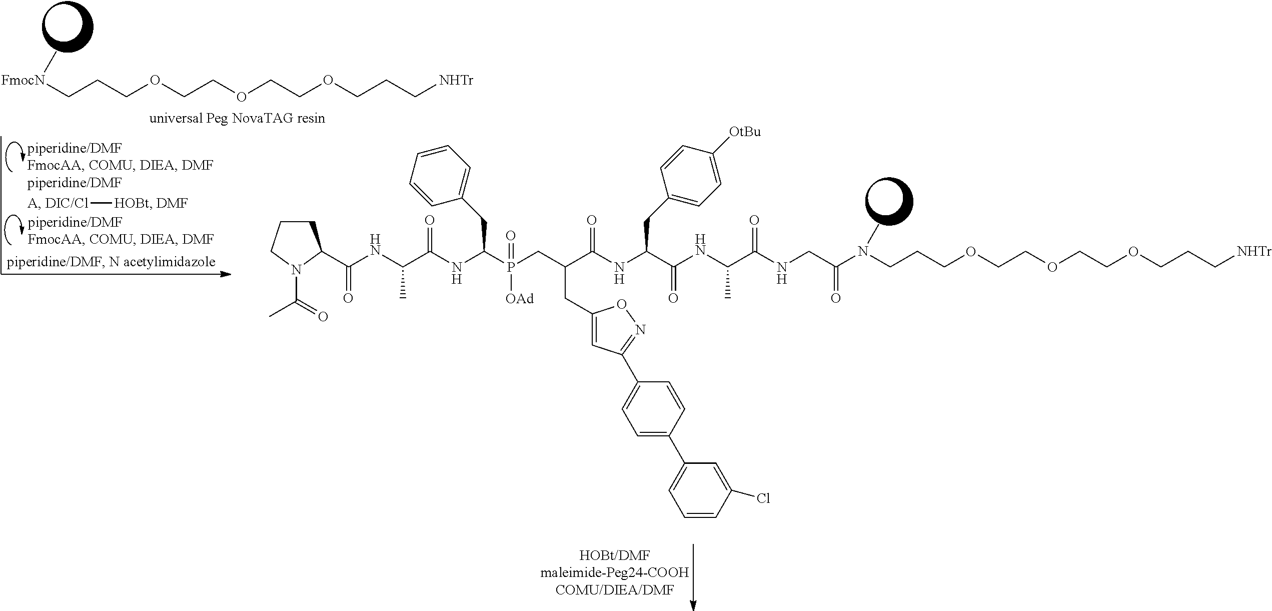

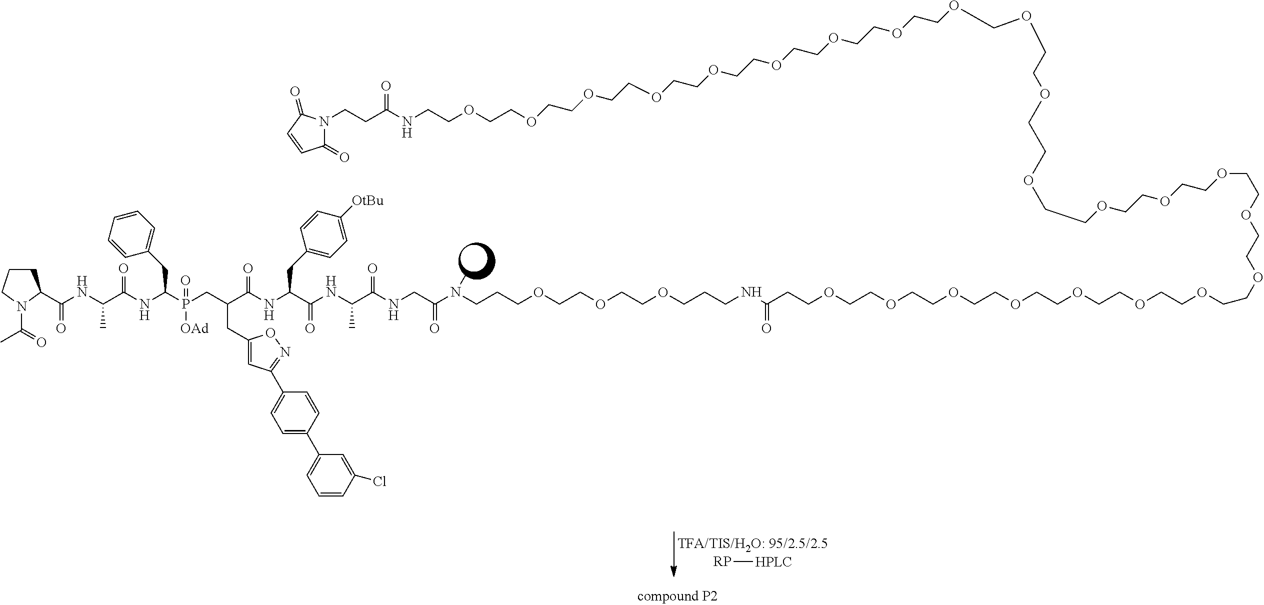

Synthesis Strategy for Ligand P3

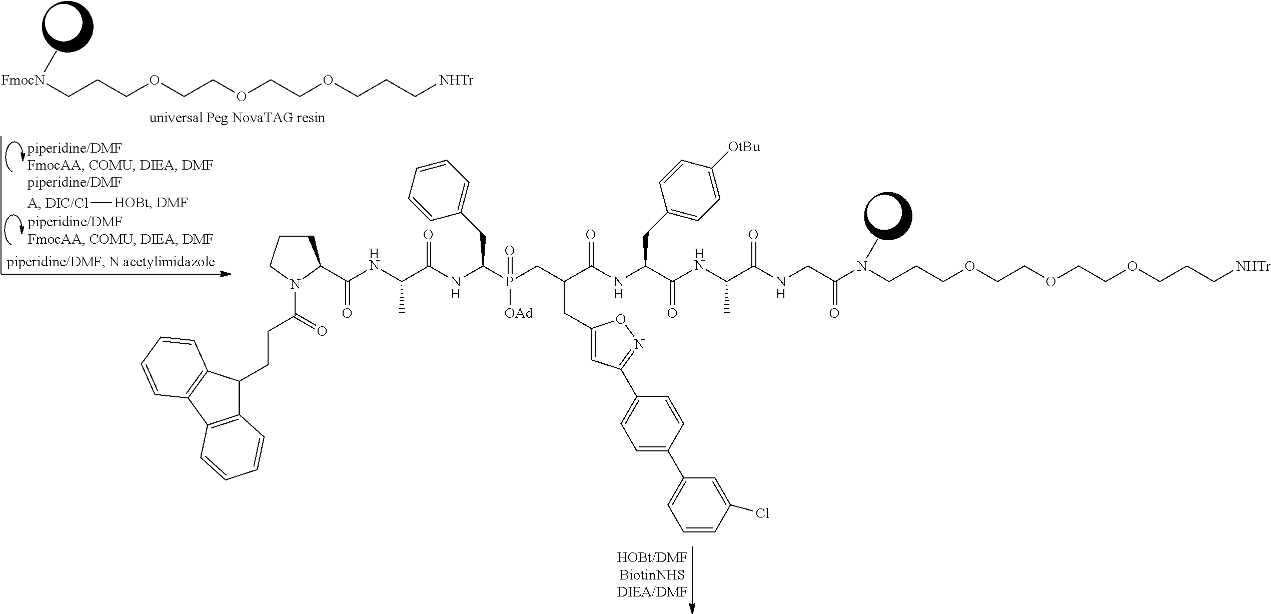

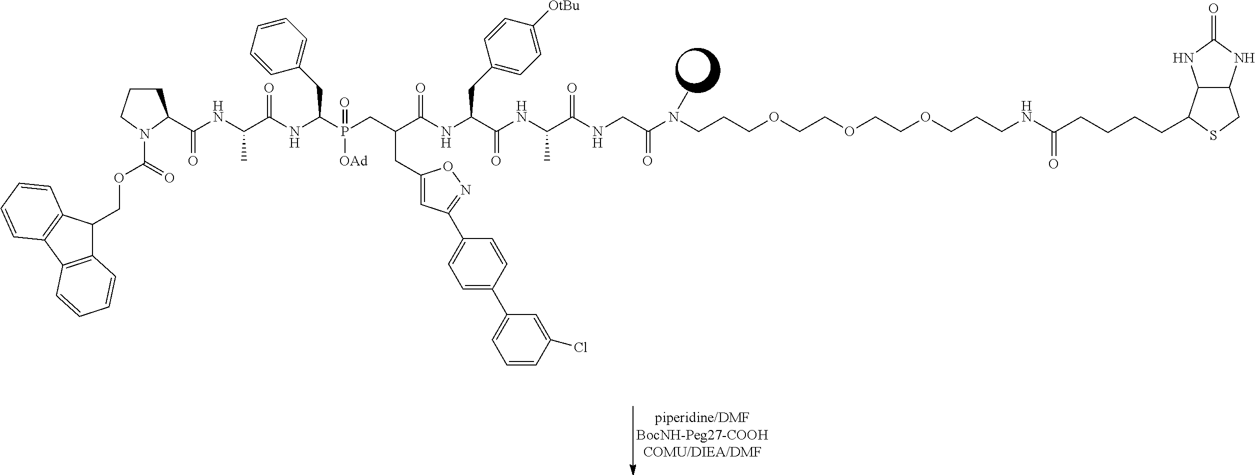

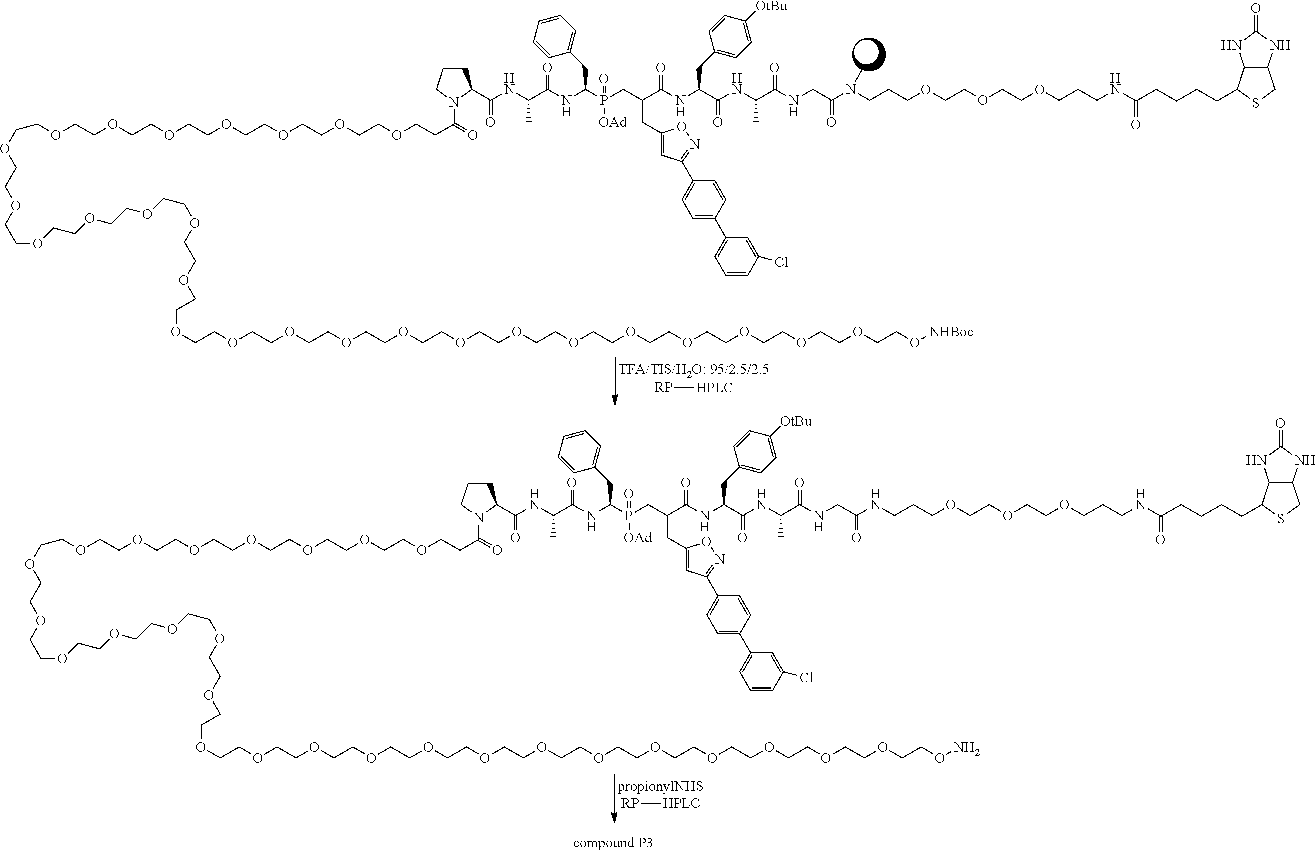

All the commercially available reagents and solvents were used as received, without additional purification. The solid support (pegNovaTag resin), natural amino acids protected by the Fmoc group, activated biotin in the form of succinimide, and COMU coupling agent used for probe synthesis were obtained from Novabiochem. The additional polyethylene glycol linkers came from Iris bitotech. The anhydrous N,N-dimethylformamide (DMF) was provided by Fluka. The 6-chloro-1-hydroxybenzotriazole (ClHOBt) and diisopropylcarbodiimide (DIC) used for incorporation of the phosphine block were respectively provided by companies Molekula and Aldrich. Trifluoroacetic acid (TFA) and triisopropylsilane (TIS) were obtained from Aldrich. BSA (bovine serum albumin) was obtained from Merck, and the HSA (human serum albumin) came from Aldrich.

Analytical and preparative RP-HPLC separations were respectively performed on a Shimadzu separation instrument and a Gilson instrument, using either an Ascentis Express analytical column (100×4.6 mm, 100 Å) or a Kromasil AIT C18 semi-preparative column (250×20 mm, 10 μm, 100A) at respective flow rates of 1, 2 and 3 mL. min⁻¹. Detection was performed at 230 nm and 275 nm. A solvent system consisting of (A) 0.1% TFA in 90% water-10% acetonitrile and (B) 0.09% TFA in 90% acetonitrile-10% water was used. The retention times (tR) obtained in analytical mode (Ascentis Express column) are given in minutes.

Optical density measurements (OD) of purified compounds P1, P2, and P3 were performed using a Beckman DU640B spectrophotometer. The recording of mass spectra involves co-precipitation of the sample with a volume of matrix 4-HCCA (Cyano-4-hydroxycinnamic acid) at 10 mg/ml in a 1/1/0.01 mixture of water/acetonitrile/TFA. The mass spectra of compounds P1, P2 and P3 were recorded using a MALDI-TOF 4800 mass spectrometer (Applied Biosystems, Foster City, USA) in negative ion reflectron mode in m/z range 800-3000. Each spectrum is the result of 1000 to 2000 shots (20 different positions within each spot and 50 shots per sub-spectrum) and an internal calibration using standard Calmix kits sold by Applied Biosystems (MDS Sciex).

Synthesis of compounds P1, P2 and P3 was performed independently and manually on solid support using:
Universal PegNova Tag resin containing a PEG (polyethylene glycol) spacer protected by a trityl group,
natural amino acids protected by the N-terminal Fmoc functional group as well as by a tertiary butyl moiety in the case of tyrosine,
PEG linkers whose terminal amine function is protected by a Boc moiety or functionalized with a maleimide.

These syntheses also use a common synthetic phosphine block: block A having a carboxylic functionality (COOH), an amine function protected by a Fmoc moiety (Fmoc), a phosphoryl functional group protected by an adamantyl moiety (Ad), and a side chain incorporating an isoxazole heterocyclic system and two phenyl moieties of which one contains a chlorine atom.

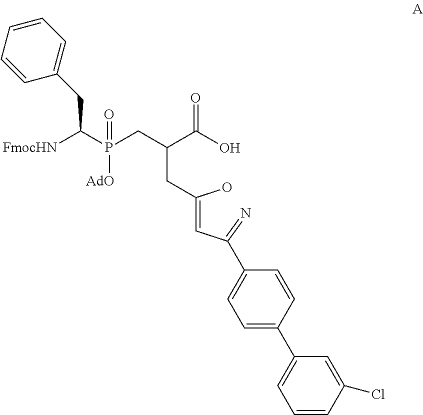

The coupling agent used to assemble these units was COMU, except for the block A coupling which used a DIC/Cl-HOBt mixture. During coupling, excess amounts of natural amino acids were used (5 eq. relative to the number of amine functions of the resin used) in the presence of DIEA (10 eq.) in DMF. The coupling of block A involved using this block with an excess of 1.5 eq in DMF. The coupling of the PEG linkers involved using 3 eq. of these linkers in the presence of six equivalents of DIEA in DMF. Capping steps (trapping the unreacted free amines) were performed after each coupling, with a solution of acetylimidazole. Removal of the N-terminal Fmoc moiety from the supported peptides or pseudo-peptides was performed using a mixture of DMF/piperidine (1/1) followed by washes with DMF and dichloromethane. Deprotection of the trityl moiety carried by the PEG linker functionalizing the resin was performed on a solid support using a solution of 0.6 M HOBt in a 1/1 mixture of TFE/CH₂Cl₂. Removal of the Boc moities carried by the PEG linkers and the protecting moities of tyrosine and block A was done during separation of the pseudopeptide from the resin, involving treating the resin with a 95/2.5/2.5 mixture of TFA/TIS/H₂O. The resulting mixtures were then purified by HPLC and resulted in compound P2 and precursors of compounds P1 and P3, each isolated in pure form. The obtained precursors of compounds P1 and P3 were respectively coupled in DMF in the presence of DIEA with succinimide derivatives of acetylthioacetate and propanoic acid, respectively yielding the pure compounds P1 and P3 after removing the excess succinimide reagent by HPLC.

The purities of compounds P1, P2, and P3 were documented by the spectra obtained from analytical RP-HPLC. The spectra showed a single peak, and the retention time in minutes for a gradient of 10 min. from 0 to 100% B were 6.65 for P1, 6.4 for P2, and 6.4 for P3.

Compounds P1, P2 and P3 were characterized by mass spectrometry using negative ion reflectron mode.

The inhibition constants Ki of compounds P1, P2, and P3 were determined in comparison to human MMPs 2, 3, 8, 9, 12, 13, 14 and to murine MMP12. Recombinant proteases were used in volumes of 100 μL at concentrations of 0.05, 0.3, 0.03, 0.05, 0.03, 0.015, and 0.5 nM respectively for human MMPs 2, 3, 8, 9, 12, 13, 14, and at a concentration of 0.1 nM for murine MMP12. The Ki determinations in this test used a commercial fluorogenic substrate MCA-Mat at 15 μM following a procedure analogous to the one described in Devel et al, (2006, J. Biol Chem, 281 (16):11152-60). Recall that the lower the Ki of a compound, the greater its potential for inhibiting the chosen target. The Ki and the amounts of enzyme used in each inhibition test are listed in the table below.

| MMP | | 2 h | 3 h | 8 h | 9 h | 12 h | 13 h | 14 h | 12 m |
|---|---|---|---|---|---|---|---|---|---|
| nM | | 0.05 | 0.3 | 0.03 | 0.05 | 0.03 | 0.015 | 0.5 | 0.1 |
| Ki | P1 | 0.12 | 0.75 | 0.09 | 0.28 | 0.03 | 0.017 | 0.53 | 0.08 |
| nM | P2 | 0.15 | 1 | 0.12 | 0.48 | 0.05 | 0.03 | 0.7 | 0.09 |
| | P3 | 0.3 | 1.55 | 0.47 | 0.73 | 0.26 | 0.07 | 1.82 | 0.66 |

### Functionalized Proteins

### Functionalized Serum Albumines

Ligand P1 was used to polyfunctionalize BSA to yield BSA-P1, while ligand P2 was used to monofunctionalize HSA or BSA respectively yielding HSA-P2 or BSA-P2.

Preparation of BSA Polyfunctionalized with Ligand-P1: BSA-P1

BSA-P1 was obtained from coupling ligand P1 having a SATA moiety with BSA functionalized with maleimide moieties. The presence of these moieties on BSA was obtained by coupling a heterofunctional linker (N-succinimidyl-4-(N-maleimidomethyl)-cyclohexane-1-carboxylate: SMCC) to commercial BSA.

Coupling of ligand P1 containing one SATA to BSA-SMCC was performed (molar ratio of inhibitor/BSA-SMCC equal to 10) after deprotecting the thiol functional group of ligand P1 by adding 1M hydroxylamine solution pH 7 for 30 minutes at room temperature.

The resulting solution was used without further purification for:
its absorption on strips used for the ICT technique, or at the bottom of the wells of the plates used for EIA method.
preparations providing the OR-BSA-P1 tracer.

Preparation of HSA Monofunctionalized with Ligand-P2: HSA-P2

HSA-P2 was obtained by coupling ligand P2 containing a maleimide moiety with HSA.

Commercial HSA isolated from human plasma was placed beforehand under controlled reductive conditions to achieve complete reduction of the cysteine at position 64 of the HSA, initially in the form of a mixture of reduced form (SH=free thiol) and oxidized thiol (adduct with endogenous cysteine from plasma). This treatment, performed using a 2 mg/ml HSA solution in the presence of 2 molar equivalents of DTT in 100 mM sodium phosphate buffer pH 6, was followed by removal of the reducing agent using a 10 kDa Filtron. The resulting mixture was placed with a molar equivalent of ligand P2 to yield HSA-P2. The resulting solution was used without further purification for:
its absorption on strips used for the ICT technique, or at the bottom of the wells of the plates used for EIA method.
preparations providing the biotin-HSA-P2 or OR-HSA-P2 tracers.
   The same protocol is used to prepare BSA-P2.

### Functionalized AChE(G4)

0.1 nmol AChE(G4-SMCC), tetrameric form of acetylcholinesterase (available from SPI-BIO) was coupled by thioether linkage to 2.7 nmol P1 to obtain AChE(G4)-P1, the AChE polyfunctionalized with ligand P1. 10 μL of 1M hydroxylamine solution was added to 2.7 nmol P1 in 40 μl of 100 mM sodium phosphate buffer pH 6 EDTA 5.10 M. After 30 minutes of incubation at room temperature, the solution containing P1 was added to 62.5 μL of 100 mM sodium phosphate buffer pH 6 EDTA 5.10 containing 0.1 nmol AChE (G4-SMCC). The whole was stirred overnight at 4° C. and then a purification step on Biogel A015M (size exclusion gel) using a buffer of 100 mM phosphate pH 7.4, 0.4 M NaCl, 0.5% BSA in the presence of NaN₃ allowed to isolate a stock solution of AchE(G4)-P1 tracer having activity of 368 EU/min/ml. This purification step was monitored by quantitation of the fractions collected using acetylthiocholine and DTNB.

### Immunochromatographic Test on Strips (ICT)

### Strip Preparation

Conducting the ICT involved preparing strips functionalized with BSA-P1, HSA-P2, or mAb12.

The functionalized strips (final dimensions: 0.5 cm×4.5 cm) were prepared using two components: nitrocellulose membrane (reaction area) and nitrocellulose blotting (adsorption area facilitating migration by capillarity).

The reaction area was used for immobilization of two types of entities immobilized in two stripes:
test stripe or test area,
antibody migration control stripe resulting from immobilization of anti-mouse immunoglobulins at 800-100 μG/mL in PBS (CAS) or anti-rabbit immunoglobulins (SAL).

The test stripe or test area corresponded to immobilization of:
serum albumin functionalized with ligand P1 or P2 (BSA-P1 or HSA-P2) or
a monoclonal or polyclonal anti-MMP12h antibody of interest.

The test stripes were deposited by hand with a 12×0.3 μL comb applicator from the Phastsystem (Pharmacia) electrophoresis system at a rate of 2 μL/cm on the sample deposit area, on the basis of 200 ng/cm solution for BSA-P1 and 2 mg/cm for HSA-P2 or the monoclonal or polyclonal antibodies.

The control stripe resulted from immobilization of CSA or SAL deposited at a rate of 1 μl/cm of a solution in PBS using an automated dispenser (BioDot AirJet XYZ 3050), respectively at 0.1 mg/mL and 0.5 mg/mL.

Following these deposits, the membranes were dried in an oven at 40° C. for 1 hour. They were then incubated twice for 30 mM at room temperature (20° C.) under slow oscillation in a saturated solution composed of 10 mM sodium phosphate buffer pH 7.4 containing 0.15 M NaCl and 0.5% BSA in the presence of NaN₃. These incubations were intended to block any residual binding sites. The membranes were then washed with ultrapure water (Milli-Q H₂O) and then incubated for 30 minutes at room temperature in a solution of 10 mM sodium phosphate buffer pH 7.4 containing 0.15 M NaCl and 0.1% Tween 20. The membranes were then drained on absorbent paper towel and dried in an oven for 15 min. at 40° C., then the membranes for sample deposit and absorption were glued to the bottom and top of the nitrocellulose membrane before being cut into strips 5 mm wide using a programmable automated cutting machine (Bio-Dot CM-400 Guillotine). The BSA-P1, HSA-P2, mAb strips so obtained were stored in plastic containers at room temperature and away from light.

### Preparation of OR-BSA-P1, OR-HSA-P2, OR-BSA-P2, and OR-mAb Tracers

Obtaining OR-BSA-P1, OR-HSA-P2, OR-BSA-P2, and OR-mAb12 tracers required preparing a stock solution of colloidal gold. This solution was obtained by adding, while stirring constantly, 4 mL of 0.2% gold chloride (AuCl₂) solution to a boiling solution of ultrapure water (40 mL MilliQ H₂O) to which 1 mL of 1% sodium citrate solution had been previously added. After the solution turned “wine-colored” (subsequent to a black color), the return of the resulting solution to room temperature was performed. The stock solution was stored at 4° C. and away from light.

To 25 μg antibody or functionalized serum albumin (in max 50 μL) in solution in a buffer of 10 mM sodium phosphate pH 7.4, 0.15 M NaCl, 0.01% NaN₃, was added 1 mL of colloidal gold stock solution and 100 μl of 20 mM borax buffer (trisodium citrate) pH=9. After one hour of incubation under mechanical rotation (4° C.), 100 μl of a buffer of 20 mM borax pH=9 1% BSA was added to the mixture, followed by centrifugation at 15000 g for 50 minutes at 4° C. Following centrifugation, the supernatant was removed and the pellet taken up in a buffer of 1 mL 2 mM borate, 1% BSA, 0.01% azide. After resuspension of the pellet, the resulting solution was centrifuged at 15000 g for 50 minutes at room temperature. After removing the supernatant the pellet was taken up in a buffer of 2504, 2 mM borate, 1% BSA, 0.01% NaN₃. The OR-BSA-P1, OR-HSA-P2, OR-BSA-P2, and OR-mAb12 gold tracers were kept at a final concentration of 100 μg/mL and stored at 4° C., away from light.

### Conducting the ICT

The use of strips functionalized with BSA-P1 or HSA-P2 was paired with visualization by OR-mAb12 tracers, OR-polyAb12. The use of strips functionalized with the antibody was paired with visualization by OR-BSA-P1, OR-HSA-P2, or OR-BSA-P2 tracers.

The test was performed at room temperature in a 96-well microtiter plate. 100 μL of native sample, diluted or reconstituted, or 50 μL of a sample were placed in a well respectively empty or containing a buffer of 50 μL 100 mM Tris-HCl, pH 6.8, 0.5% Tween-20, 1% Chaps, 0.1% NaN₃ already present in the well.

Strips Functionalized with BSA-P1 or HSA P2 Paired with Visualization by OR-mAb12, OR-polyAb12 Tracers

After absorption of the solution contained in the wells, the strips were washed using a buffer of 100 mM Tris-HCl pH 7.4, 0.5% Tween-20, 1% Chaps, 0.1% NaN₃ or 50 mM Tris-HCl pH 6.8, 10 mM CaCl₂, 0.01% Brij. The volumes and number of repetitions of these washes differed according to whether or not the treated sample is a sample containing biological media. Washing with 50 μL, then 30 μL, then μL 20, was performed in the case of analysis of a sample containing biological medium, while a 30 μL wash followed by a 20 μL wash was performed for samples containing only recombinant proteins (with or without addition of competitor). Following the addition of 20 μL, a volume of 5 to 10 μL of tracer was added to the well when the tracer was the monoclonal antibody. Tracer migration allowed visualization of the protein captured by either the BSA-P1 or HSA-P2, by focusing a signal visible to the naked eye along the deposited test stripe. The intensity of the observed signal was estimated qualitatively by the naked eye.

Strips Functionalized with Antibodies Paired with Visualization by OR-BSA-P1, OR-HSA-P2, or OR-BSA-P2 Tracers.

In this case, the tracer may or may not be incubated with the sample from the start. After stirring the mixture, the strip was inserted vertically into the well near the sample deposit area. The liquid was wicked up the strip by capillarity. Migration of the tracer allowed visualization of the protein captured by the monoclonal antibody, by focusing a signal visible to the naked eye along the deposited test stripe. The intensity of the observed signal was estimated qualitatively by the naked eye.

### Enzyme Immunoassay (EIA)

Preparation of Plates Functionalized with BSA-P1, HSA-P2, mAb12, or polyAb12

Plates bearing BSA-P1, HSA-P2, or the monoclonal antibody were prepared using plates commonly used for immunoassays. 100 μL of solutions at 2 μg/mL (BSA-P1), 5 μg/mL (HSA-P2), and 5 μg/mL (the monoclonal antibody—mAb, or polyclonal antibody—polyAb) in 50 mM sodium phosphate buffer pH 6 were used to functionalize the wells of the plates. After overnight incubation at 4° C. and removal of the supernatant, 300 μL of 10 mM sodium phosphate buffer solution pH 7.4 containing 0.15 M NaCl and 0.1% BSA, 0.1% NaN₃ were added to the wells to block any residual binding sites. The plates were stored in this buffer at 4° C. until use.

### Tracer Preparation:

1—Biot-mAb12 and biot-HSA-P2 Tracers

Biot-mAb12 and Biot-HSA-P2 were prepared from a solution of mAb12 or HSA-P2 in 100 mM borate buffer pH 9 and from a solution of active biotin in the form of N-hydroxysuccinimide ester (biotin NHS) at 5 mg/mL in freshly prepared DMF.

Biot-mAb12: 250 μg of mAb12 (125 μL of a solution at 2 mg/mL in PBS) were placed in 200 μL of 100 mM Borate buffer pH=9.0, followed by the addition of biotin-NHS to 20 μL at 5 mg/mL. After incubation for 45 minutes at room temperature, 115 μL of 1M Tris buffer pH=8.0 was added to the mixture. Incubation for 10 minutes was followed by the addition of 650 μL of a buffer of 100 mM phosphate pH 7.4, 0.1% BSA, 0.15 M NaCl, 0.01% NaN₃ EIA (0.1% BSA) to achieve a concentration of 200 μg/mL Biot-mAb12 tracer. The Biot-mAb12 stock solution was stored at 4° C.

Biot-HSA-P2: 175 μg of HSA-P2 (87 μl of a 2 mg/mL solution in PBS) were placed in 200 μL of 100 mM Borate buffer pH=9.0 followed by the addition of 9 μL biotin-NHS at 5 mg/ml. After incubation for 45 minutes at room temperature, 100 μL of 1M Tris buffer pH=8.0 was added to the mixture. Incubation for 10 minutes was followed by the addition of 475 μL of a buffer of 100 mM phosphate pH 7.4, 0.1% BSA, 0.15M NaCl, 0.01% NaN₃ EIA to achieve a stock concentration of Biot-HSA-P2 tracer of 200 μg/mL. The Biot-HSA-P2 stock solution was stored at 4° C.

2—P3 Tracer

The P3 ligand was used as is once isolated after purification after being dissolved DMSO.

3—AChE(G4)-P1 Tracer

The AChE(G4)-P1 tracer was used as is once isolated after purification.

### Conducting the EIA

The use of plates functionalized with BSA-P1 or HSA-P2 was paired with visualization via biot-mAb12h tracers.

The use of plates functionalized with antibodies (mAb12 or polyAb12) was paired with visualization via biot-HSA-P2, P3 or AChE(G4)-P1 tracers.

After leaving the functionalized plates at room temperature, the wells were washed with an automatic plate washer in a cycle of 5 washes, by a volume of 300 μL using a buffer of 10 mM phosphate, 0.1% Tween, pH 74.

In the case of EIA assays using Biot-mAb12h tracer or biot-HSA-P2 tracer (in a sequential version), samples 100 μL in volume in varying buffers were then placed in the wells for incubation of either 4 hours or overnight. This step, corresponding to the step of capturing the species of interest in the sample, was followed by removal of the supernatant and a series of washes with soaking for 2 to 5 minutes while stirring, using a buffer of 100 mM Tris HCl, 10 mM CaCl₂, 1 M NaCl, 0.5% Tween. This was followed by the addition of biotinylated tracer at a concentration of 1 μg/mL used in a buffer of PBS pH 7.4, 0.15 M NaCl, 0.1% BSA, 0.01% NaN₃ for biot-mAb12 and in a buffer of 50 mM Tris HCl, pH 6.8, 10 mM CaCl₂, 0.01% Brij for biot-HSA-P2. Incubation with the tracer was conducted overnight at 4° C. before removal of the supernatant, and a further series of washes with a buffer of 50 mM phosphate, 0.1% Tween 20. 1 EU/min/ml of streptavidin AchE(G4) (acetylcholinesterase) was added, followed by incubation for 2h before removal of the supernatants, washes, and addition of acetylthiocholine and DTNB (DTNB=5×10⁻⁴ M, acetylthiocholine 1.4×10⁻⁵ M in 10 mM phosphate buffer). Detection of the protein of interest was measured by measuring the activity of AChE(G4) whose action on its substrate in the presence of DTNB provides an absorbance at 414 nm.

In the case of EIA assays using Biot-HSA-P2 tracer (in a simultaneous version), the sample was pre-incubated with biot-HSA-P2 then placed into the plates. After capture of the protein of interest by the grafted antibody and washes, 1 EU/min/mL of streptavidin AChE(G4) was incubated, then, after removing the supernatant, detection of the protein of interest was carried out by adding acetylthiocholine and DTNB and measuring the absorbance produced at 414 nm.

In the case of EIA assays using P3 tracer, the sample was preincubated with a concentration of 20 or 100 nM of P3, then placed in the plates. After capture of the protein of interest by the grafted antibody (mAb12h plate, polyAb12m plate, polyAb12h plate), 1 EU/min/mL of streptavidin AChE(G4) was incubated, then, after removing the supernatant, detection of the protein of interest was carried out by adding acetylthiocholine and DTNB and measuring the absorbance produced at 414 nm.

In the case of EIA assays using AchE(G4)-P1 tracer, the sample was preincubated on a plate with 2 EU/min/ml AChE(G4)-P1. After capture of the protein of interest by the grafted antibody (mAb12h plate for example) or by the antibody in solution used with CAS or SAL plates, after removing the supernatant, detection of the protein of interest was carried out by adding acetylthiocholine and DTNB and measuring the absorbance produced at 414 nm.

Results

### Immunochromatographic Test on Strips (ICT)

### BSA-P1 Strips: Results

The BSA-P1 strips correspond to strips on which 100 ng BSA polyfunctionalized with ligand P1 are immobilized. The use of BSA-P1 strips was paired with the use of OR-mAb12h tracer (monoclonal anti-human MMP12 antibody).

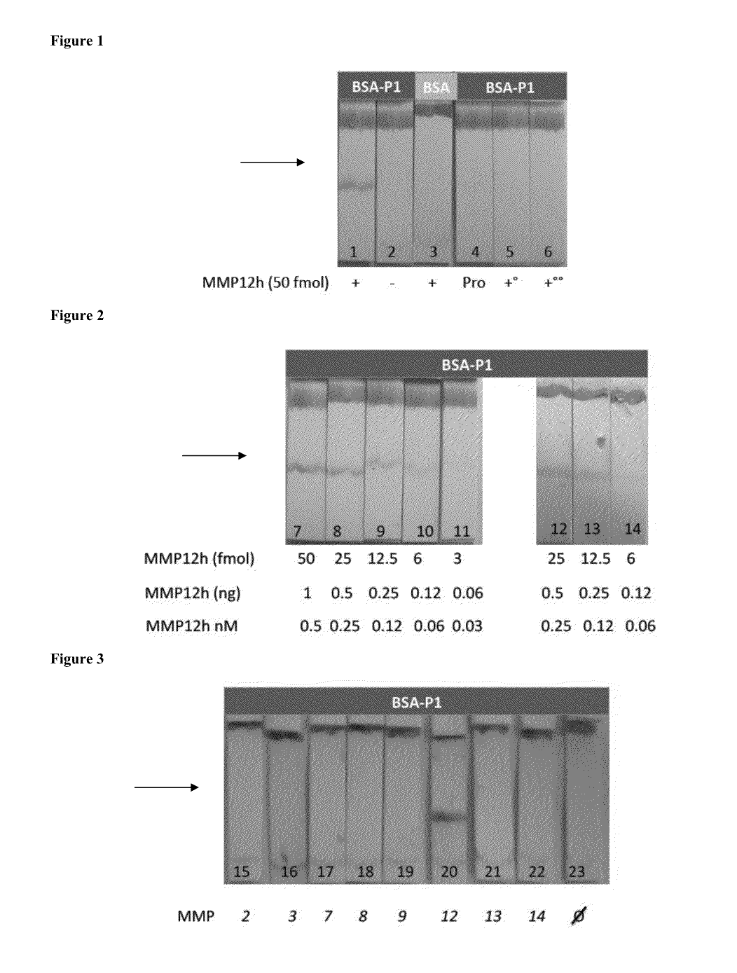

Detection of Human MMP12 (MMP12h)—[FIG. 1]

BSA-P1 strips paired with the use of OR-mAb12h tracer enable positive detection of MMP12h (1) via visualization of a signal visible to the naked eye in the central BSA-P1 immobilization area on the strip. This detection is specific to the use of MMP12h in the experiment since, in the absence of MMP12h (2), migration of OR-mAb12h tracer along the strip resulted in the absence of any observable signal in the BSA-P1 immobilization area on the strip. Positive detection of MMP12h (1) results from an interaction between MMP12h and ligand P1 carried by BSA-P1. Indeed, migration of MMP12h and OR-mAb12h tracer along a strip carrying immobilized unmodified BSA does not result in observing a positive signal (3), while the migration control at the top of the strip ensures effective migration of the OR tracer. Positive detection of MMP12h (1) implies interaction of MMP12h at its active site, given that the use of MMP12h solution previously incubated with a phosphinic inhibitor (competitor) specific for MMP12h having a Ki of 0.2 nM with respect to MMP12h (referred to as “Compound 1” or RXP470.1 and described in Devel et al, 2006, J Biol Chem, 281, 11152-11160) at a concentration of 100 nM)(°) or 1 μM (°°) does not result in observing a signal (respectively 5, 6) in the BSA-P1 immobilization area. Similarly, proMMP12 corresponding to MMP12h inhibited by its protein precursor is not detected by this system (4).

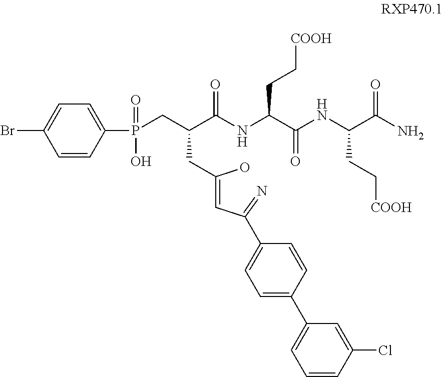 [SMILES: N=C(/C=C(\O)C[C@H](CP(=O)(O)c1ccc(Br)cc1)C(=O)N[C@@H](CCC(=O)O)C(=O)N[C@@H](CCC(=O)O)C(N)=O)c1ccc(-c2cccc(Cl)c2)cc1]

All these experiments allowed concluding that using BSA-P1 strips in combination with OR-mAb12h tracer allows detecting MMP12h at the BSA-P1 immobilization site via the formation of a ternary complex involving, on the one hand, molecular interactions between the active site of MMP12h and the ligand functionalizing BSA, and on the other hand molecular interactions between a domain of MMP12h and the anti-MMP12h monoclonal antibody bound to the gold tracer (OR-mAb12h). Only the MMP12h providing a free active site, and therefore active, can be detected, the MMP12h proform and a form previously inactivated by an MMP12h inhibitor are not detected. This system is therefore well-suited for specifically detecting the active form of MMP12h.

Sensitivity of MMP12h Detection in Buffered Medium—[FIG. 2]

The utilization of a range of concentrations of MMP12h in a buffer of 50 mM Tris-HCl pH 6.8, 10 mM CaCl₂, 0.01% Brij, and BSA-P1 strips paired with the use of OR-mAb12h showed that the developed system allows detection of MMP12h using buffered samples containing up to 6 fmol MMP12h, which is equivalent to a weight of 0.12 ng or a concentration of 0.06 nM (10). However, using this buffer produces non-uniform migration effect as is reflected by the wavy appearance of the revealed stripe. Using a range of concentrations of MMP12h in a 50/50 mixture of 50 mM Tris-HCl pH 6.8, 10 mM CaCl₂, 0.01% Brij and of 100 mM Tris-HCl pH 7.4, 0.5% Tween-20, 1% Chaps, 0.1% NaN₃ allowed to rectify this effect, and in this case the detection threshold reached a value between 6 and 12.5 fmol (between 0.12 and 0.25 ng, or a molar concentration between 0.06 nM and 0.12 nM) as shown by strips (13) and (14).

Specificity of the System for Detection of MMP12h Versus Other MMPs—[FIG. 3]

The selectivity of the system for detection of MMP12h versus other MMPs has been verified by experiments using independent solutions of MMP-2, -3, -7, -8, -9, -12, -13, and -14. These experiments confirmed the selectivity of the monoclonal antibody although the MMPs used, which share a high homology, could have been captured by the P1 ligand functionalizing the BSA immobilized on the strip. Indeed, after migration of the MMPs and the OR-mAb tracer, the only positive signal was observed from the MMP12h solution (20), while the other MMP solutions provide no visible signals on the strips (15, 16, 17, 18, 19, 21, 22), also not detected when MMP12h is absent (23).

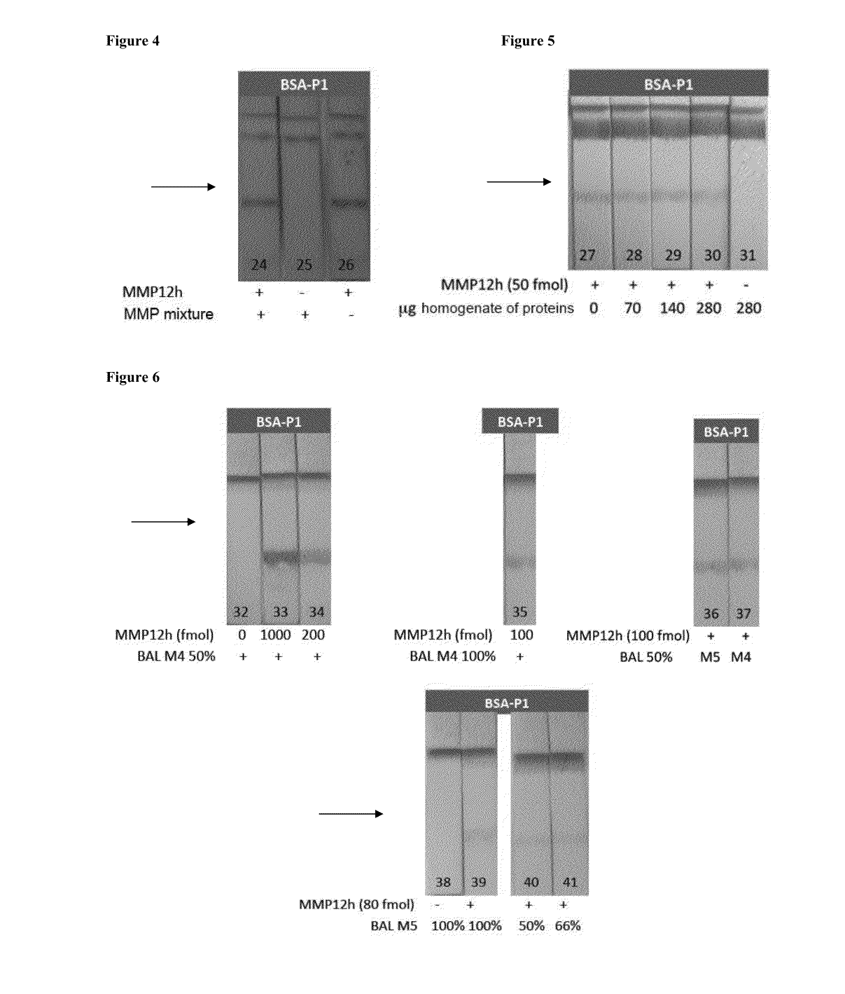

Specificity of the System for Detection of MMP12h when Other MMPs are Present —[FIG. 4]

The ability of the system to detect only MMP12 in a mixture of MMPs has also been tested, by comparing experiments using solutions containing different MMPs (2, 9, 13, 14) including MMP12 (24) or not including it (25) and a solution containing only MMP12h (26). No signal at the BSA-P1 immobilization site on the strip is visible with the MMP mixture (25), while a signal is detected with the mixture including MMP12h (24). This signal has an intensity comparable to that obtained with an MMP12h solution in buffered medium (26).

Detection in a Complex Environment

Homogenate of Cytosolic Proteins—[FIG. 5]:

When 50 fmol MMP12h are added to 70, 140, or 280 μg of a complex mixture of cytosolic proteins, MMP12h was detected by the system of BSA-P1 strips paired with OR-mAb12h by a signal (28, 29, 30) of intensity comparable to that observed when the MMP12h used is in the presence of buffer (27). The signal observed with protein mixtures containing added MMP12h does indeed originate from the MMP12h, given that 280 μg of homogenate of various proteins used did not produce an observable signal at the BSA-P1 immobilization site on the strip (31). Thus, the use of BSA-P1 strips in combination with visualization by OR-mAb12h is possible with complex protein mixtures and in the case of cytosolic proteins provides a positive detection signal when MMP12h is present at only 0.0003%.

Bronchoalveolar Lavages (BAL) [FIG. 6]:

The possibility of detecting MMP12h using the system of BSA-P1 strips combined with OR-mAb12h tracer was also tested by adding MMP12h to mouse bronchoalveolar lavages. These experiments were conducted to characterize the compatibility of a such a system for detecting MMP12h in human BAL. With this in mind, two types of media were evaluated: named BAL M4 and BAL M5. 100% BAL M4 corresponds to a mouse BAL obtained in PBS and diluted to half with a buffer of 50 mM Tris-HCl pH 6.8, 10 mM CaCl₂, 0.01% Brij. 100% BAL M5 corresponds to a mouse BAL obtained in PBS diluted to half with 50 mM Tris-HCl pH 6.8, 10 mM CaCl₂, 0.01% Brij, then again diluted to half in 50 mM Tris-HCl pH 6.8, 10 mM CaCl₂, 0.01% Brij, 1M NaCl, 2M urea. When the indicated percentages are less than 100%, the 100% BAL M4 and 100% BAL M5 were diluted before migration in the wells of the microtiter plate, with the following buffer 100 mM Tris-HCl pH 7.4, 0.5% Tween-20, 1% Chaps, 0.1% NaN₃. The results of experiments conducted using bronchoalveolar lavages showed that detection of MMP12h is possible in these media. Indeed, while the BAL M4 and BAL M5 media alone resulted in no signal at the BSA-P1 immobilization site (32, 38), the presence of a signal allowed detecting a picomole (33), 200 fmol (34), and 100 fmol (35) of MMP12h, whether the BAL M4 medium was used as is (35) or diluted to 50% with 100 mM Tris-HCl pH 7.4, 0.5% Tween-20, 1% Chaps, 0.1% NaN₃ (33, 34). Detection of 100 fmol MMP12h is compatible with the presence of urea and NaCl in the medium (at respective concentrations of 1M and 500 mM). In fact, the detection signal for 100 fmol MMP12h added to 100 μl of 100% BAL M5 (36) is comparable to the one for 100 fmol MMP12h added to 100 μl of 100% BAL M4 (37). The detection signal for 80 fmol MMP12h added to 100 μl of 100% BAL M5 (39) is comparable to the one for 80 fmol MMP12h added to 100 μl of 50% BAL M5 (40) and 66% BAL M5 (41).

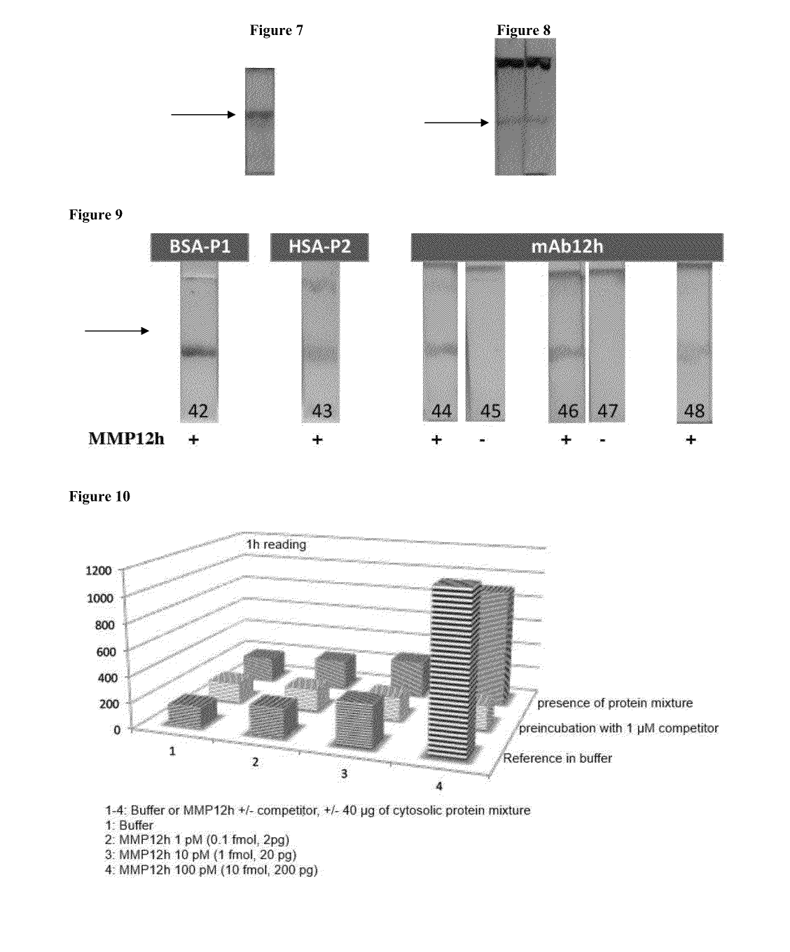

Applicability of the System for Detection of Other MMPs—[FIG. 7, FIG. 8]

The system has been tested for detection of active human MMP13. The tracer used is a monoclonal anti-human MMP13 antibody with a strip loaded with BSA-P1.

A positive signal was observed when using only active MMP13h in the experiment (FIG. 7). The system was tested for detection of active murine MMP12 (FIG. 8). The tracer used was a polyclonal anti-murine MMP12 antibody. A positive signal is observed when using only active MMP12m in the experiment, in a buffered medium or in the presence of 40 μg of cytosolic protein extracts. These results demonstrate the functionality of the system for capturing MMPs of different types, which are then detectable by means of an antibody specific for the MMP concerned.

### Other Strips: Results

In addition to BSA-P1 strips, other types of strips produced were the strips referred to as HSA-P2 strips and mAb12h strips. These strips correspond to strips on which HSA monofunctionalized with ligand P2, and monoclonal antibody mAb12h were respectively immobilized. Similarly to BSA-P1 strips, the use of HSA-P2 strips is paired with the use of OR-mAb12h tracer. The use of mAb12h strips is paired with the use of OR-BSA-P1 or OR-HSA-P2 tracers or even OR-BSA-P2 (gold tracer of BSA functionalized with ligand P2).

Comparison of Detection Efficiency of MMP12h by BSA-P1, HSA-P2, and mAb12h Strips—[FIG. 9]

The same buffered MMP12h solution was used with different strips respectively visualized by the corresponding tracer. A positive signal is observed regardless of the type of strip involved in the experiment when MMP12h is used in the experiment (42, 43, 44, 46 and 48), indicating that:
the HSA-P2 immobilized on the strip is able to capture the MMP12h used in the experiment and visualized at the HSA-P2 immobilization site on the strip by OR-mAb12h tracer (43);
OR-BSA-P1 (44), OR-HSA-P2 (46), and OR-BSA-P2 (48) are tracers capable of detecting MMP12h captured by mAb12h immobilized on the strips.

These results provide evidence that the inhibiting part consisting of ligand P1 or P2 of the BSA or HSA can be a partner in the ligand/MMP/mAb ternary complex, whether the serum albumin is immobilized on the strip or constitutes the tracer. With mAb12h strips, the observed signal indicating MMP12h detection is of comparable intensity whether the serum albumin forming the tracer is polyfunctionalized (44) or monofunctionalized (46) and whether the serum albumin is human (46) or bovine (48). Therefore when serum albumin is used as a tracer, the degree of functionalization of the serum albumin by the ligand of the MMP of interest has negligible effect on the ability of the tracer to detect MMP12h captured by the mAb12h immobilized on the strip. On the other hand, this degree of functionalization appears to have an effect on the ability of serum albumin immobilized on strips to capture the MMP12h used in the experiment. Indeed, the intensity of the signal observed with an HSA-P2 strip paired with the use of OR-mAb12h (43) is significantly lower than that observed with the same MMP12h solution and a BSA-P1 strip paired with the use of OR-mAb12h (42). Given the same tracer being used in both cases (42, 43) and the comparable inhibitory capacities of ligands P1 and P2, these results indicate an intensified capture capacity for strips prepared with polyfunctionalized serum albumin solution (BSA-P1) compared to strips prepared with monofunctionalized serum albumin solution (HSA-P2). In addition, the signal observed on the BSA-P1 strip by visualization with OR-mAb12h (42) is stronger than those observed with the same MMP12h solution using strips with mAb12h, whether the tracer used corresponds to OR-BSA-P1 (44), OR-HSA-P2 (46), or OR-BSA-P2 (48).

### Summary of Results for the Strip Format

The system of strips containing immobilized BSA-P1 paired with the use of mAb12h tracer allows selective and specific detection in buffered medium of 6 fmol (0.12 ng) of MMP12h having one active site free (active form of MMP12h) per 100 μl sample, which is a detection sensitivity of 1.2 ng/mL. Detection of MMP12h only works in this system if and only if the MMP12h presents a free active site, unlike its proform with pro-peptide or its form inactivated by an inhibitor. Indeed, the system does not give a positive signal when ProMMP12h and an MMP12h/inhibitor complex are used in the analysis. Among the MMPs tested, only MMP12h generates a positive signal and this signal can be observed even when the MMP12h is initially in a mixture with other MMPs having high homology. The system is compatible with detection of MMP12h (1 ng) that is initially in a protein rich medium (240 μgrams), which represents a possibility for detecting MMP12h when the latter represents 0.003% of the total proteins. In addition, the developed system is compatible with detection of MMP12h placed in mouse bronchoalveolar lavages (BAL). To date, the detection sensitivity demonstrated in these environments is 80 fmol per 100 ul of BAL, or 16 ng/mL. The developed strips are therefore also compatible with complex media such as cell extracts or bronchoalveolar lavages. It should be noted that strips in ICT format offer an advantage as a method for detecting an active MMP of interest in a biological sample because the strips are stable over time and provide a rapid analysis.

Serum albumin functionalized with the ligand of the MMP of interest can be used for detecting MMPs, both as an entity immobilized on strips for capturing a target MMP subsequently visualized via a monoclonal antibody (42, 43, FIG. 9), or as a tracer able to reveal the presence of the MMP of interest captured by a monoclonal antibody immobilized on strips (44, 46, 48, FIG. 9). The degree of functionalization of the serum albumin by the ligand has little impact on the ability of the functionalized serum albumin to detect captured MMP12h, while the degree of functionalization has significant impact on the ability of the serum albumin to capture MMP12h for subsequent visualization. This effect has a positive correlation with increases in the degree of functionalization. Finally, the format providing the highest detection sensitivity corresponds to the format based on pairing the use of serum albumin polyfunctionalized with the ligand of the target MMP immobilized on the solid support, and a tracer consisting of a monoclonal antibody labeled with gold.

### Strip Format Conclusion

The strip format offers the greatest sensitivity for exclusively detecting active MMP when serum albumin polyfunctionalized with a ligand of the MMP of interest is used as the agent for capturing, concentrating, and focusing the MMP of interest on the strip, its presence being subsequently visualized by a labeled monoclonal or polyclonal antibody, for example colloidal gold. However, serum albumin monofunctionalized with a ligand of the MMP of interest is also effective for capturing the MMP of interest, but with lower sensitivity. In addition, the serum albumin mono- or polyfunctionalized with a ligand of the MMP of interest may have value as a tracer when it is labeled, particularly with colloidal gold, and used with strips functionalized with the antibody specific for the target MMP.

EIA Test

The possibility of transferring the approaches explored in ICT format (strips) has been evaluated in designing EIA tests that would result in a positive signal solely in the case where the MMP of interest is present in the active form, in other worlds having the active site unoccupied. For this approach, several types of plates were prepared:
BSA-P1 plate, with immobilization of BSA polyfunctionalized with ligand P1;
HSA-P2 plate, with immobilization of HSA monofunctionalized with ligand P2;
mAb12h plate, polyAb12h plate, and polyAb12m plate, respectively with immobilization of monoclonal anti-human MMP12 antibody, polyclonal anti-human MMP12 antibody, and polyclonal anti-murine MMP12 antibody.

The use of BSA-P1 plates and HSA-P2 plates was paired with detection using a monoclonal antibody labeled with biotin (Biot-mAb12h), with secondary detection by streptavidin-AChE(G4) capable of cleaving acetylthiocholine in the presence of DTNB, which generates an absorbance signal at 414 nm.

The use of mAb12h plates, polyAb12m plates, and polyAb12h plates was respectively paired with detection using:
Biot-HSA-P2, P3 or AChE(G4)-P1 in the case of mAb12h plates;
P3 in the case of polyAb12h plates and polyAb12m plates.

In all cases, the signal measured is the absorbance signal at 414 nm generated by AchE(G4) cleaving acetylthiocholine in the presence of DTNB. The data provided below are the results, for each measurement point, of at least three runs of experiment.

BSA plate P1: Results

Detection Sensitivity in Simple and Complex Media—[FIG. 10] The use of a BSA-P1 plate combined with detection via Biot-mAb12 resulted in detection of MMP12h, whether the MMP12h was in a buffered medium of 50 mM Tris-HCl 6.8, 10 mM CaCl₂, Brij 0.01% or in the presence of 40 μg of a mixture of cytosolic proteins in the same buffer. The MMP12h detection is positive up to initial concentrations of 1 pM in buffered medium and between 1 and 10 pM in a complex medium consisting of 40 μg of a mixture of cytosolic proteins, which respectively represent initial quantities of MMP12h in an analysis volume of 100 μL of 0.1 fmol (which is 2 pg) and between 0.1 and 1 fmol (which is between 2 and 20 pg).

The specificity of the MMP12h capture via its unoccupied active site (therefore only the active form of MMP12h) by the ligand P1 carried by BSA has been verified by eliminating the generation of absorbance when the analyzed sample contains MMP12 previously inhibited in solution, in simple or complex medium, by an MMP inhibitor (competitor).

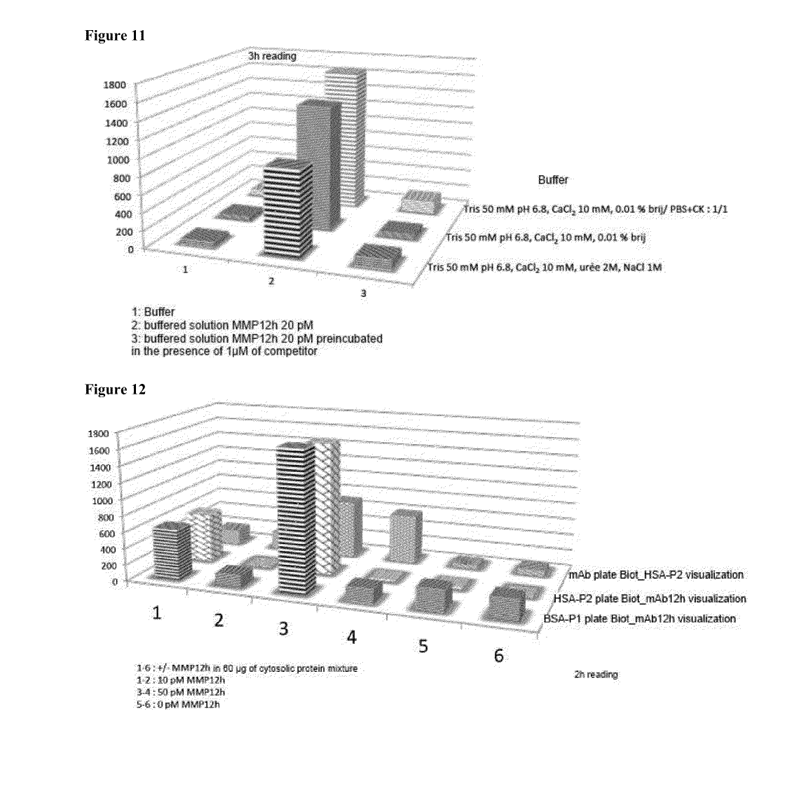

Compatibility with Different Buffers—[FIG. 11]

The compatibility of the BSA-P1 plate system combined with detection via Biot-mAb12h was established for various buffers normally used as diluents for biological media or tissue extraction solutions. The absorbances obtained were compared by analyzing solutions having a volume of 100 μl, of 20 mM MMP12h in:
a buffer of 50 mM Tris-HCl pH 6.8, 10 mM CaCl₂, 0.01% Brij; or
in a buffer mixture of 50 mM Tris-HCl pH 6.8, 10 mM CaCl₂, 0.01% Brij/PBS: 1/1 containing a cocktail of protease inhibitors except for metalloproteinase inhibitors; or
in a buffer of 50 mM Tris-HCl pH 6.8, 10 mM CaCl₂, 2M urea, 1M NaCl.

Compatibility of the MM12h detection system with these different types of buffered media was observed, as well as in presence of a mixture of organic molecules constituting the protease inhibitor cocktail. The lack of absorbance generated when MMP12h has previously been inhibited in solution by an MMP inhibitor (competitor) indicates the specificity of the observed signals, since only MMP12h with an unoccupied active site (active form) is detected in the experiment.

The absorbances generated have similar but not identical intensities. The signal measured for an initial MMP12h concentration of 20 pM is greater in a mixture of 50 mM Tris-HCl pH 6.8, 10 mM CaCl₂, 0.01% Brij/PBS (1/1) containing a cocktail of protease inhibitors (except metalloproteinase inhibitors) compared to that observed in a buffer of 50 mM Tris-HCl pH 6.8, 10 mM CaCl₂, 0.01% Brij, the latter being higher than that observed in a buffer of 50 mM Tris-HCl pH 6.8, 10 mM CaCl₂, 2M urea, 1M NaCl. These intensity differences may reflect the variable capacity of the MMP12h depending on the buffer to maintain its three-dimensional structure as well as the capacity of the ligand P1 carried by the BSA to interact effectively with its target.

Comparison of BSA-P1 Plates, HSA-P2 Plates, and mAb12h Plates for MMP12h Detection in Complex Media: Results—[FIG. 12]

In these experiments, samples were incubated in plates, then removal of the supernatant was followed by washes and the addition of the tracer. Using identical solutions of MMP12h at 10 or 50 pM in the presence of 40 μg of a mixture of cytosolic proteins for detection of MMP12h using BSA-P1, HSA-P2, and mAB12h plates indicates that:
MMP12h is detected by both the BSA-P1 plate//Biot-mAb12h tracer system and the HSA-P2 plate//Biot-mAb12h tracer system, indicating that in the case of an EIA format, the degree of substitution of serum albumin has no significant effect on the quality of the MMP12h detection. However, it should be noted that in this experiment, the samples were incubated overnight which can encourage optimal capture in terms of quantity of the species of interest.
the HSA-P2//Biot-mAb12h tracer system detects, in complex media, 1 fmol of MMP12h per 100 μl sample, which corresponds to an initial MMP12h concentration of 10 pM.
similarly to the BSA-P1//Biot-mAb12h tracer system, the HSA-P2//Biot-mAb12h tracer system does not detect MMP12h when access to its active site is blocked beforehand by a competitor, rendering the system specific for detection of MMP12h having its active site unoccupied (MMP12h in active form).
the background noise generated by BSA-P1 plates for a sample of complex proteins is greater than that observed by HSA-P2 plates (comparison of fields 5 and 6). This greater noise can also be seen in experiments where samples are preincubated with a competitor (fields 2 and 4).

On the other hand, the mAb12h plate/Biot-HSA-P1 tracer system when used sequentially stands out from the other two systems in the fact that:
the detection sensitivity is lower. Indeed, when MMP12h solution is used at a concentration of 10 pM in 40 μg of a mixture of cytosolic proteins, the absorbance observed with the mAb12h plate/Biot-HSA-P1 tracer does not allow concluding a positive detection of MMP12h because the signal intensity is comparable to that observed in the absence of MMP12h.
when access to the active site of the MMP12h was previously prevented by a competitor, the MMP12h is still detected (field 4) by a signal intensity comparable to that obtained with MMP12h solution in the absence of a competitor (field 3). The explanation for this detection of previously inactivated MMP12h probably lies in the competitor being removed by washes after capture of the MMP12h.

The mAb12h plate/Biot-HSA-P1 tracer system, when only the incubation with the plate and the tracer are sequential, thus allows detection of MMP12h but without being selective for MMP12h in its active form (in other words having its active site unoccupied).

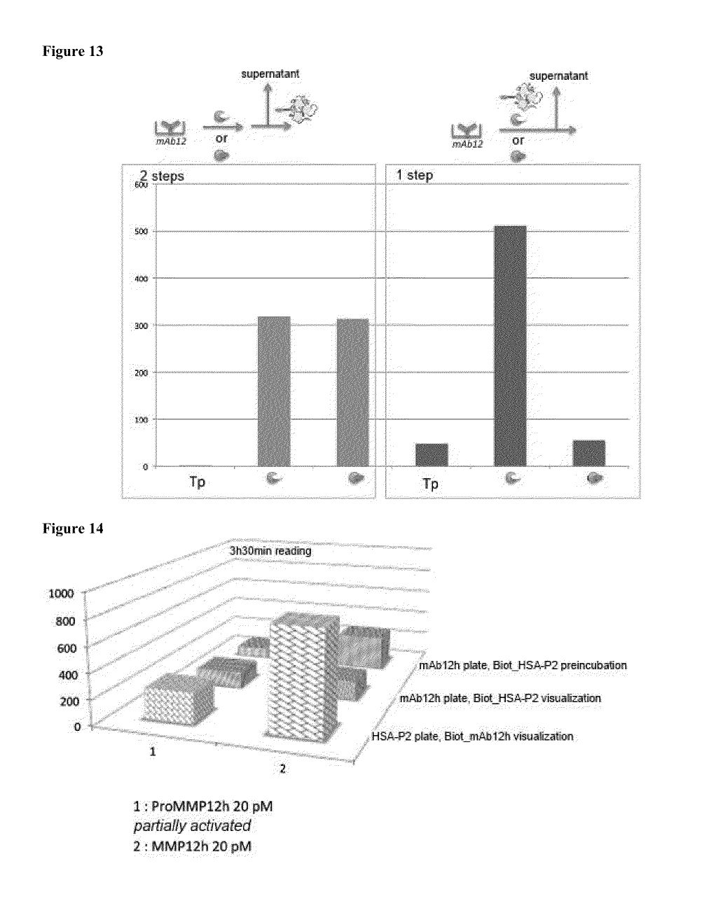

Comparison of Times when Biot-P2-HSA Tracer is Added in Assays Using mAb12h Plates for MMP12h Detection: Results—[FIG. 13]

In these experiments, the samples were incubated in plates in the presence or absence of Biot-HSA-P2 tracer, then removal of the supernatant was followed by washes and the addition of Biot-HSA-P2 tracer when the latter was not present during sample incubation.

The results show that when the plates are functionalized with an antibody, solely a one-step protocol (the sample containing the MMP to be detected and the tracer were incubated together from the outset in a well functionalized with antibody capable of recognizing the MMP to be detected) enables clear discrimination between the presence of active forms and initially inactivated forms (complexed with an inhibitor) of MMP12h. In a two-step protocol (biot-HSA-P2 tracer is added after incubation of the sample containing MMP and removal of the supernatant), a positive detection signal is obtained whether the MMP is initially active or is complexed with an inhibitor. Detection of only the active form of MMP therefore requires all reactants to be incubated together from the outset, to allow quantitation of active MMP via the measured absorbance as proportional to the amount of biot-HSA-P2//active MMP complex.

This system has been validated for detection of MMP12h (50 pM) using plates loaded with mAb12h and for detection of MMP12m (50 pM) using plates loaded with polyAb12m.

Comparison of HSA-P2 Plates and mAb12h Plates for Specific Detection of Active MMP12h Relative to ProMMP12h: Results [FIG. 14]

Solutions of MMP12h and ProMMP12h (proform of MMP12h self-inhibited by its own protein precursor) at 20 pM in a buffer of 50 mM Tris-HCl pH 6.8, 10 mM CaCl₂, 0.01% Brij were used for HSA-P2 plate//Biot-mAb12h tracer systems and mA12h plate/Biot-HSA-P2 tracer systems to assess the ability of these systems to exclude detection of ProMMP12h relative to detection of MMP12h.

In the case of the use of mAb12h plates, two types of tracer addition times were once again evaluated. As above, one case involves addition of the tracer following the capture step, after removal of the supernatant and washes (mAb12h plate, Biot-HSA-P2 visualization), while the second results from incubating the sample from the outset with the Biot-HSA-P2 tracer (mAb12h plate, Biot-HSA-P2 preincubation). The results indicate, as previously observed, that MMP12h is better detected by the system using an HSA-P2 plate than by the system using a functionalized mAb12h plate, regardless of when the tracer is added.

On the other hand, MMP12h detection appears more effective for mAb12h plates when the tracer is incubated with the sample from the outset (field 2, mAb12h plate, Biot-HSA-P2 preincubation). In addition, this one-step incubation protocol is also better at excluding detection of ProMMP12, whose associated signal is weaker when Biot-HSA-P2 tracer is present during sample incubation on the mAb12h plate.

As for the analysis of ProMMP12h solution by HSA-P2 plate, the signal intensity tends to indicate detection of MMP12h from this sample. It can therefore be inferred that the ProMMP12h solution also contains a portion of activated MMP12h. Measurements of activity of the solutions used with a fluorogenic substrate explained these results. Indeed, these measurements indicated that the ProMMP12h solution used contained an estimated proportion of 20% MMP12h capable of degrading the fluorogenic substrate. Thus, the ProMMP12h sample actually corresponds to a mixture of ProMMP12h having its active site occupied, and MMP12h where the unblocked active site gives this species the ability to degrade a substrate or to interact with the P2 inhibitor carried by the HSA immobilized on the HSA-P2 plates. The HSA-P2//Biot-mAb12h tracer system is able to detect this proportion of active MMP12h in the ProMMP12h sample. The HSA-P2 plate//Biot-mAb12h tracer system is thus able to distinguish the presence of MMP12h and ProMMP12h by providing a positive detection signal solely in the case of solutions containing active MMP12h.

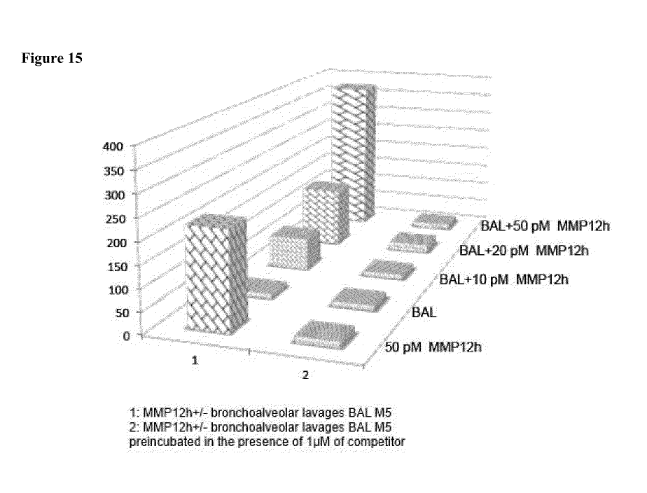

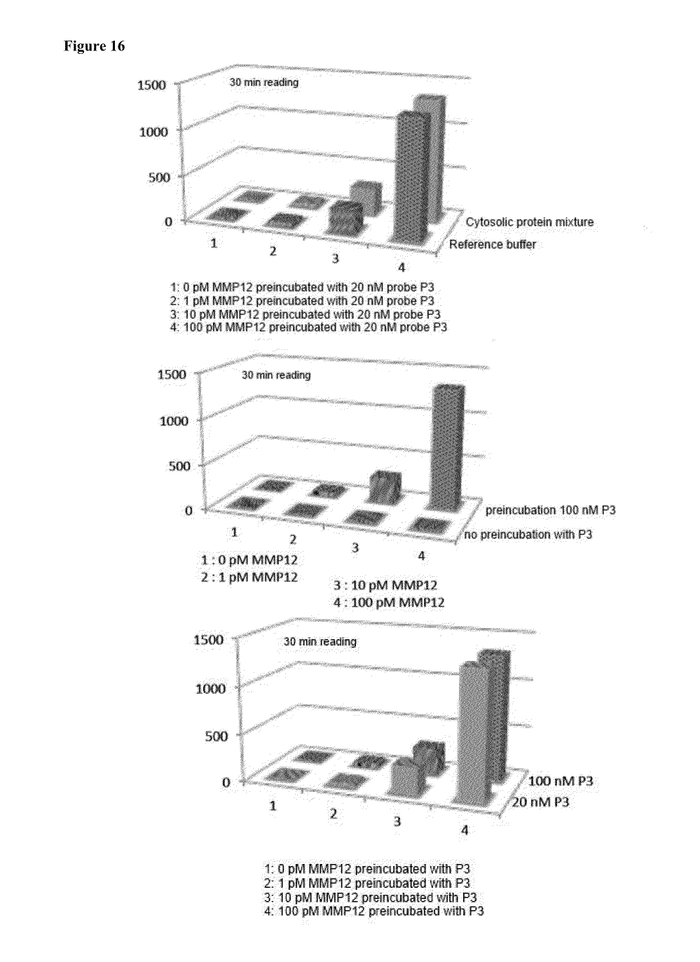

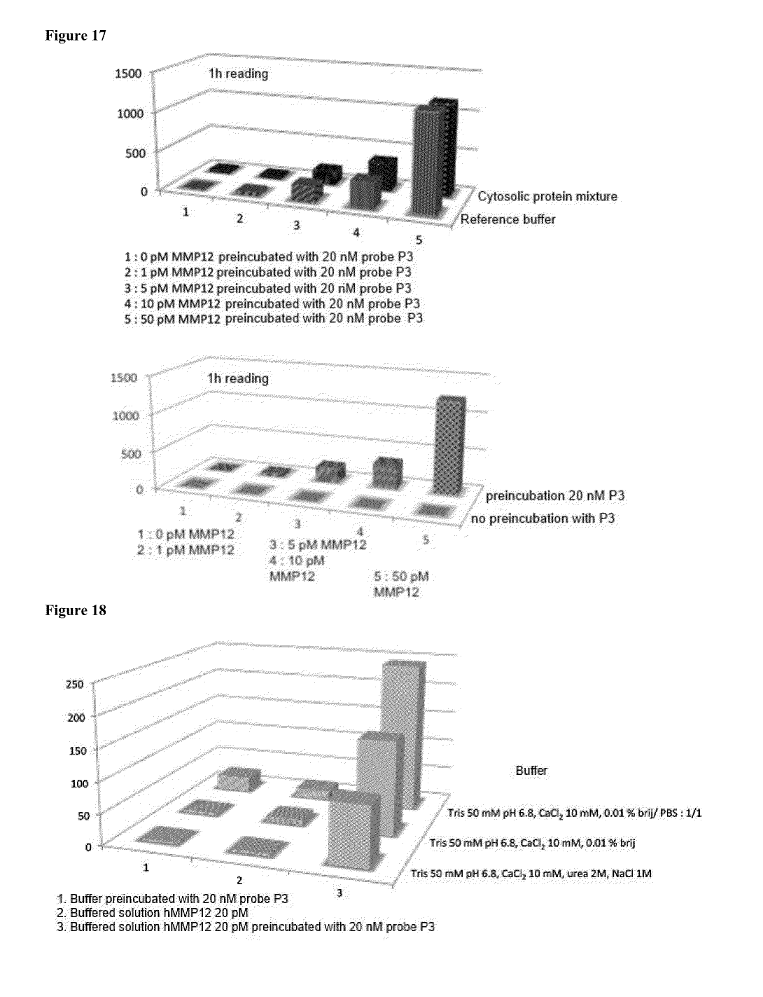

HSA P2 Plates for Detection of MMP12h in Bronchoalveolar Lavages: Results—[FIG. 15]

The possibility of detecting MMP12h with the system pairing the HSA-P2 plate with use of Biot-Ab12h tracer was also tested by adding different amounts of MMP12h to mouse bronchoalveolar lavages. These experiments were planned in order to characterize the compatibility of such a system for detecting MMP12h from human BAL. With this in mind, 100% BAL M5 was used. This medium is mouse BAL obtained in PBS diluted to half in 50 mM Tris-HCl pH 6.8, 10 mM CaCl₂, 0.01% Brij, then again diluted to half in 50 mM Tris-HCl pH 6.8, 10 mM CaCl₂, 0.01% Brij, 1M NaCl, 2M urea.

The results indicate compatibility of the HSA-P2/Biot-Ab12h tracer system for MMP12h detection when proteins are present in the BAL. The signals observed (field 1) are suppressed if the samples are preincubated with a competitive inhibitor targeting the active site of the MMP12h of interest.

The detection sensitivity in the BAL medium used was 1 fmol of MMP12h per 100 μL of sample, which represents a molar concentration of 10 pM and a mass concentration of 200 pg/mL.

The same result profiles are obtained when the plates used are functionalized with anti-MMP12h polyclonal antibodies (polyAb12h plate) in the presence of MMP12h. The tracer was ligand P3 which contains a biotin part. The use of mAb12h immobilized on the plate in this strategy achieves the same levels of sensitivity as using polyAb12h.

This system is compatible with incubations performed in various types of buffer such as 50 mM Tris-HCl pH 6.8, 10 mM CaCl₂, 0.01% Brij, or a mixture of 50 mM Tris-HCl pH 6.8, 10 mM CaCl₂, 1/1 0.01% Brij/PBS containing a cocktail of protease inhibitors except for metalloproteinase inhibitors, or 50 mM Tris-HCl pH 6.8, 10 mM CaCl₂, 2M urea, 1M NaCl.

The system is therefore compatible for detection of MM12h diluted by various types of buffered media.

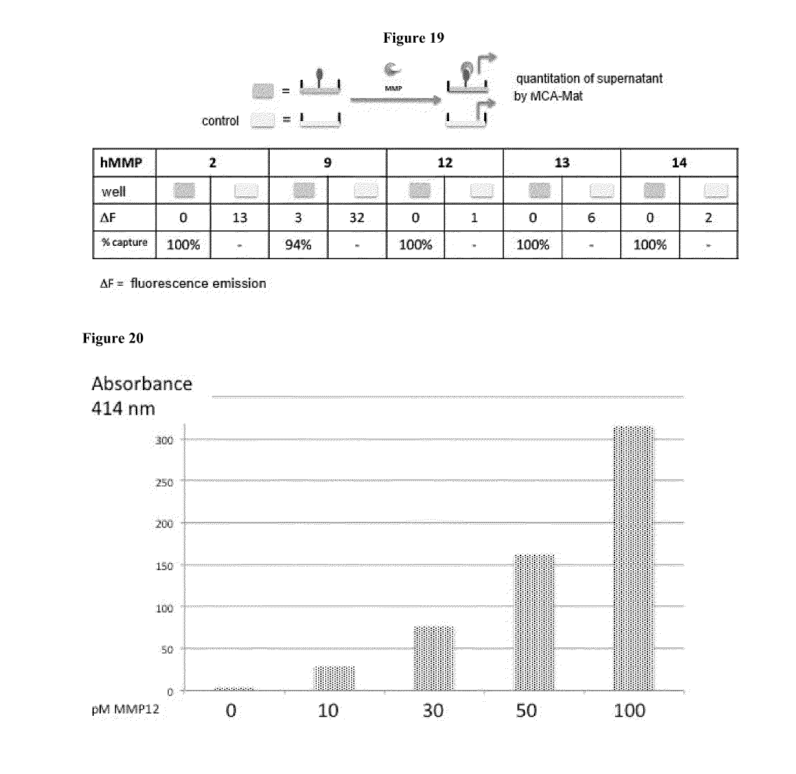

Capture of Other MMPs: Results [FIG. 19]

The ability of the EIA version of the system to capture multiple MMPs has been demonstrated by enzymatic assay of post-capture supernatants. This assay is only possible in this format (it is not possible in strip format) and functions by adding the isolated supernatant after incubation of the commercial fluorogenic substrate MCA-Mat whose cleavage in buffered medium by MMPs generates a fluorescence signal. The fluorescence measurement is expected to be zero if the MMP was captured by the entity loaded in the well (in this case serum albumin functionalized with a ligand of MMP). These measurements were carried out in comparison to supernatants originating from control wells loaded with serum albumin having no MMP ligand (control) in order to ensure that the MMP capture does indeed function via a specific interaction with the MMP ligand. These experiments were conducted with initial MMP concentrations of 100 pM, a concentration compatible with fluorescence emission (ΔF) measurable by the fluorometer used and for all MMPs tested.

The results presented in FIG. 19 clearly indicate that the capture step of the system is functional for all MMPs tested, which is a mandatory prerequisite for the detection of MMPs when using monoclonal antibodies. Thus, the captures demonstrated to be effective could be further enhanced for each MMP by using the monoclonal antibody specific for the MMP of interest to be detected.

Use of AChE(G4) Functionalized with Ligand P1: [FIG. 20]

AChE(G4-SMCC), the tetrameric form of acetylcholinesterase (commercially available from SPI-BIO) was functionalized with ligand P1 to yield AChE(G4)-P1. The binding of ligand P1 can thus be directly detected via AChE.

In a first test, all three partners, namely AChE(G4)-P1, MMP12h, and mAb12h, are incubated together in a well functionalized with CAS, an entity able to recognize the entire set of murine anti-human antibodies. After removal of the supernatant, washes, and addition of an acetylcholine substrate analog, the measured absorbance is proportional to the amount of AChE(G4)-P1//active MMP12h//mAb12h complex and thus allows quantitation of active MMP12h. This system has been shown to be effective for detection of active MMP12h up to concentrations of 10 pM (50 μL, 100 pg/mL), with a fixed amount of mAb12h (50 μL, 10 ng/mL). Similarly, this system has been validated for detection of MMP12m using a fixed amount of anti-MMP12m polyclonal antibody (immunopurified) down to concentrations of 10 pM active MMP12m (50 μL, 100 pg/mL). In this case, the plate is functionalized with SAL, an entity capable of recognizing the entire set of rabbit anti-mouse antibodies.

A variant of this system uses wells directly functionalized by antibodies specific for the MMP to be detected (rather than CAS or SAL). In this system, the AChE(G4)-P1 and the sample containing the MMP, either MMP12h or MMP12m, are incubated together from the outset in a well functionalized either by mAb or polyAb. After removal of the supernatant, washes, and addition of an acetylcholine substrate analog (AChE(G4)), the measured absorbance is proportional to the amount of AChE(G4)-P1//active MMP complex, and thus only corresponds to quantitation of the active MMP. The principle of this system has been validated for MMP12h detection (50 pM) using plates loaded with mAb12h and for MMP12m detection (50 pM) using plates loaded with polyAb12m as well as in various buffers containing no protease inhibitor cocktail.

It has been shown that this system also functions when the AchE(G4)-P1 tracer is added after incubation of the sample containing the MMP in the well. In this case, however, the detection is not specific for the active form of the MMP.

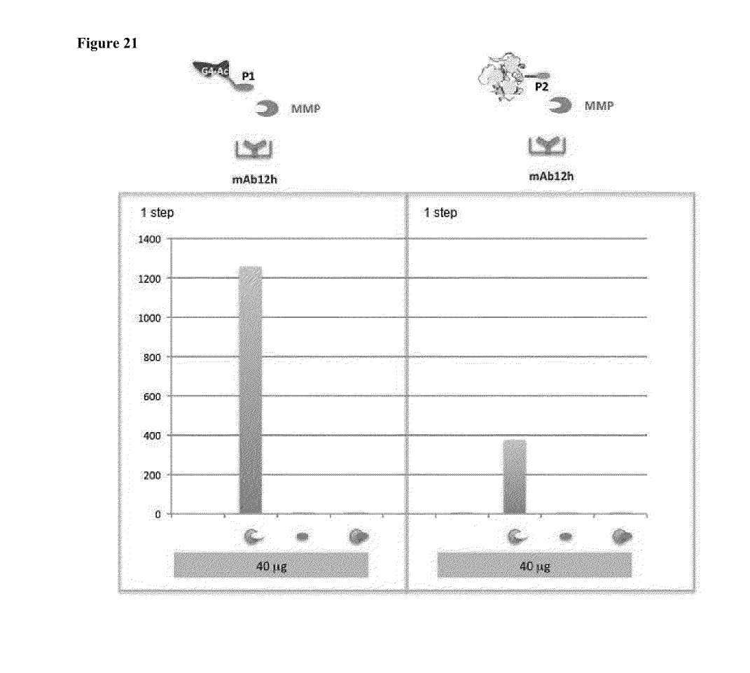

### Comparison of AchE(G4)-P1 and Biot-HSA-P2 for Detection of MMP12h in Complex Media [FIG. 21]

The use of plates functionalized with mAb12h with biotinylated serum albumin or

AChE(G4) respectively carrying ligands P2 and P1 as tracers, with simultaneous incubation of the sample with the tracer carrying ligands P1 or P2, was evaluated for MMP12h in the presence of complex media exemplified by cytosolic protein extract. Both systems resulted solely in positive detection of active MMP12h. The MMP12h initially interacting with a competitive inhibitor cannot interact with either P1 or P2 and is therefore not detected. The presence of complex media (40 μg of cytosolic protein extract) and the incubation time have no effect on the detection capacity of the system. The catalytic activity of AChE(G4) and the possibility of recognition by the streptavidin-AChE(G4) of the serum albumin carrying the ligand (in interaction with the target of interest) are not impacted by the incubation time and the presence of cell extracts having proteolytic activity. However, the signal of the system directly using AChE(G4) is higher than with the system using biot-HSA-P2 which is secondary detected by streptavidin-AChE(G4).

### Conclusion for EIA Format

In this format, serum albumin mono- and polyfunctionalized with a ligand of MMP is effective as agents for the capture, concentration of the MMP interest, the presence of the latter being subsequently revealed by a monoclonal or polyclonal antibody specific for the MMP of interest. In this case, the use of serum albumin monofunctionalized with the MMP ligand allows to reduce the cost of the system, as well as the background noise.

In EIA formats using a loaded antibody, the serum albumin and the AChE(G4)-monofunctionalized with the MMP ligand can be used as tracers for detection of active forms of MMP.

The exclusive detection of active forms of MMP in EIA format requires that the sample containing the MMP to be detected is in contact with the ligand from the outset, which is possible:
with plates functionalized with serum albumin carrying P1 or P2, or
with plates functionalized with antibodies specific for the MMP of interest provided that the sample is incubated with the MMP ligand (for example serum albumin or AChE(G4) carrying the MMP ligand) prior to or simultaneously with incubation in the wells.

These three systems have been shown to be compatible with the presence of complex mixtures of proteins, which does not affect the ability of the targeted MMP to interact with the MMP ligand carried by the carrier protein and guarantees exclusively detecting only the active forms of MMP.

It is important to note, however, that the signals obtained for MMP12h detection are systematically of higher intensity when plates functionalized with serum albumin carrying P1 or P2 are used, in comparison to the use of plates functionalized with antibodies.

MMP2h and MMP9h Detection

The compatibility of the strategy developed for MMP12 has been established for detection of other MMPs in active form, particularly human MMP2 and MMP9.

BSA-P1 plates functionalized with a ligand of the active site of MMPs were used for the capture phase and the appropriate antibody was used for the detection step. They were prepared as described above.

Biot-mAb2 and Biot-mAb9 antibodies were used. These are monoclonal antibodies functionalized with biotin molecules. Biot-mAb9 is an anti-MMP9h monoclonal antibody recognizing human MMP9. Biot-mAb2 is an anti-MMP2h monoclonal antibody recognizing human MMP2. Biot-mAb2 was obtained after a step of biotinylation of the commercial murine monoclonal antibody against human MMP2 (Anti-MMP2 (Ab-8) Mouse (VB3); Calbiochem (EMD Millipore). Biot-mAb9 corresponds to a murine monoclonal antibody against human MMP9 which is already biotinylated (MMP9 (7-11C): sc-13520, Santa Cruz Biotechnology, Inc.).

Biot-mAb2 was prepared from a solution of mAb2 in 100 mM borate buffer pH 9 and a solution of active biotin in the form of 5 mg/mL N-hydroxysuccinimide ester (biotin-NHS) in freshly prepared DMF. Specifically, 25 μg of mAb2 (25 μL of solution containing 1 mg/mL in PBS) were placed in 125 μL of 100 mM Borate buffer pH=9.0, followed by addition of 10 μl NHS-biotin at 5 mg/mL. After a 45-minute incubation at room temperature, 50 μL of 1M Tris buffer pH=8.0 were added to the mixture. Incubation for 10 minutes was followed by addition of 790 μL of a buffer of 100 mM phosphate pH 7.4, 0.1% BSA, 0.15 M NaCl, 0.01% NaN₃ to achieve a concentration of 25 μg/mL Biot-mAb2 tracer. The Biot-mAb2 stock solution was stored at 4° C.

The use of plates functionalized with BSA-P1 was combined with visualization via biot-mAb2h tracer and also with visualization via biot-mAb9h tracer for the respective detection of MMP2h and MMP9h.

After bringing the functionalized plates to room temperature, the wells were washed with an automatic plate washer in a cycle of 5 washes with a volume of 300 μL using a buffer of 100 mM phosphate, 0.1% Tween, pH 74.

In the EIA tests using BSA-P1 plates and biot-mAb2h tracer or biot-mAb9h tracer, samples of 100 μL containing increasing concentrations of active forms of MMP2h or MMP9h in solution in a buffer of 50 mM phosphate or 50 mM Tris.HCl pH 7.4, 0.1% BSA, 0.15M NaCl, 0.01% NaN₃ were then placed in the wells for incubation for 3h at 25° C. and overnight at 4° C. This step, corresponding to the step of capturing the species of interest in the sample, was followed by removal of the supernatant and a series of washes with soaking 2 to 5 minutes while stirring, using a buffer of 100 mM Tris HCl, 10 mM CaCl₂, 1M NaCl, 0.5% Tween. This was followed by addition of biot-mAb2 or biot-mAb9 biotinylated tracer at a concentration of 1 μg/mL used in a buffer of PBS or 50 mM Tris-HCl pH 7.4, 0.15M NaCl, 0.1% BSA, 0.01% NaN₃. Incubation with the tracer was conducted overnight at 4° C. before removal of the supernatant, and a further series of washes with a buffer of 50 mM phosphate, 0.1% Tween 20. 1 EU/min/ml streptavidin-AChE(G4) (acetylcholinesterase) was added followed by incubation for 2h before removal of the supernatants, washes, and addition of acetylthiocholine and DTNB (DTNB=5×10⁻⁴ M, acetylthiocholine 1.4×10⁻⁵ M in 10 mM phosphate buffer). Detection of the protein of interest was measured by measuring the activity of AChE(G4) whose action on its substrate in the presence of DTNB provides an absorbance at 414 nm.

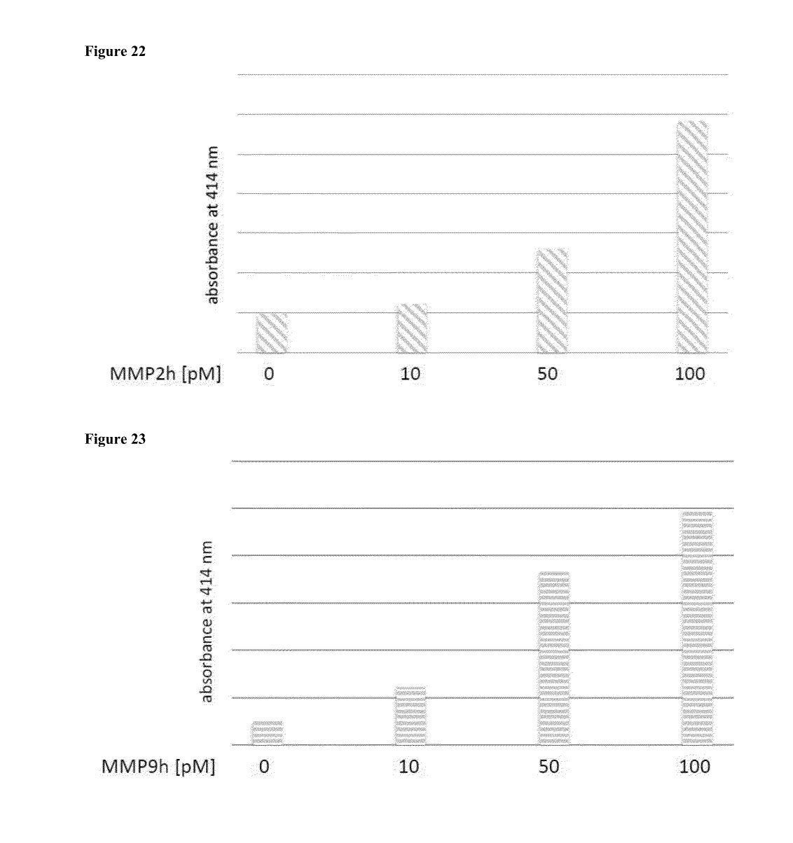

Use of the BSA-P1 plate combined with detection via Biot_mAb2h or Biot_mAb9h results in the respective detection of MMP2h (FIG. 22) and MMP9h (FIG. 23) in a buffered medium of 50 mM Tris.HCl pH 7.4, 0.1% BSA, 0.15M NaCl, 0.01% NaN₃. Detection of MMP2h and MMP9h was demonstrated as positive for initial concentrations of 10 pM in buffered medium, which respectively represents initial quantities of MMP2h and MMP9h of 1 fmol (respectively 66 pg and 65 pg) in an analysis volume of 100 μL

These results indicate that the steps of capture and positive detection of active forms of MMP are not dependent on the size and molecular weight of the MMP. Detection remains solely dependent on the type of antibody used and the presence of the MMP in its active form.

In addition to detection of MMP12, 2, or 9 in samples with comparable sensitivities at the pM level (10⁻¹² M), the use of other existing anti-MMP monoclonal antibodies, particularly those commercially available, allows providing detection systems for all MMPs.

1. An in vitro method for specifically detecting in a biological sample a matrix metalloproteinase (MMP) of interest only in its active form, comprising
a) a step of placing the biological sample in contact with a ligand of the MMP of interest capable of binding to the free active site of the MMP;
b) subsequently to or simultaneously with step a), a step of placing the result of step a) in contact with an antibody specific for the MMP of interest; and
c) a step of detecting the ternary complex between the MMP of interest, the ligand of the MMP of interest, and the antibody specific for the MMP of interest,
and wherein the ligand comprises a phosphinic pseudopeptide inhibitor.

2. Method according to claim 1, wherein either the ligand or the antibody is immobilized on a solid support, and detection is achieved either by detecting the antibody when the ligand is immobilized on the support, or by detecting the ligand when the antibody is immobilized on the support.

3. Method according to claim 2, comprising
a) providing a solid support on which a ligand of the MMP of interest is immobilized;
b) placing the solid support in contact with the sample so as to allow attachment of the MMP of interest present in the sample to the solid support via a binding between the ligand and the MMP of interest;
c) optionally, removing the unattached MMPs;
d) adding an antibody specific for the MMP of interest to allow formation of the ternary complex between the MMP of interest, the ligand of the MMP of interest, and the antibody specific for the MMP of interest;
e) optionally, removing the free antibodies; and
f) detecting antibodies in the ternary complex, this detection being indicative of the active form of the MMP of interest being present in the sample.

4. Method according to claim 2, comprising
a) providing a solid support on which an antibody specific for the MMP of interest is immobilized;
b) placing the solid support in contact with the sample which has been previously or is simultaneously placed in contact with a ligand of the MMP of interest, so as to allow attachment of the MMP of interest present in the sample to the ligand and immobilization of the MMP of interest on the solid support via a linkage between the antibody and the MMP of interest;
c) optionally, removing the free MMPs and ligands; and
d) detecting the ligand in the ternary complex between the MMP of interest, the ligand of the MMP of interest, and the antibody specific for the MMP of interest, this detection being indicative of the presence in the sample of the active form of the MMP of interest.

5. Method according to claim 1, wherein the method uses an immunochromatographic test (ICT) or a solid phase immunoassay, the solid phase possibly being a membrane (flow-through), a well of a microtiter plate (EIA for example), or strips (SPT).

6. Method according to claim 1, wherein detection of the ligand or antibody is obtained by its covalent or non-covalent coupling to a detectable marker.

7. Method according to claim 6, wherein the detectable marker is selected from the group consisting of a colloidal metal, a non-metal colloid, carbon, a visible, fluorescent, luminescent, or chemiluminescent tracer, a magnetic particle, a radioactive element, and an enzyme, preferably colloidal gold or an enzyme.

8. Method according to claim 1, wherein the ligand is coupled to a carrier protein, preferably via a linker or spacer, in particular a polyethylene glycol linker.

9. Method according to claim 8, wherein the carrier protein is mono- or polyfunctionalized with the ligand of the MMP of interest.

10. Method according to claim 8, wherein the carrier protein is serum albumin, preferably human or bovine.

11. Method according to claim 1, wherein the biological sample is a biological liquid or fluid, or a tissue or cell extract.

12. Method according to claim 1, wherein the MMP of interest is selected from among MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27, and MMP-28, preferably from among MMP-2, MMP-3, MMP-8, MMP-9, MMP-10, MMP-12, MMP-13, and MMP-14, is preferably MMP-12.

13. Method according to claim 1, wherein the ligand comprises a moiety of formula (I): 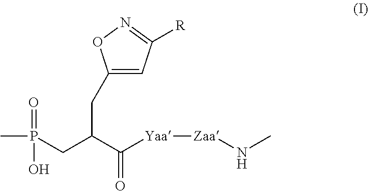
where
Yaa′ is an natural amino acid other than Asp, Pro, Gly, Cys, and Gln, in particular selected from the group consisting of Ala, Arg, Asn, Glu, His, Ile, Leu, Lys, Met, Phe, Ser, Thr, Val, Trp, and Tyr;
Zaa′ is a natural amino acid other than Pro and Cys, in particular selected from the group consisting of Ala, Arg, Asp, Asn, Gly, Gln, Glu, His, Ile, Leu, Lys, Met, Phe, Ser, Thr, Val, Trp, and Tyr;
R is selected from the group consisting of

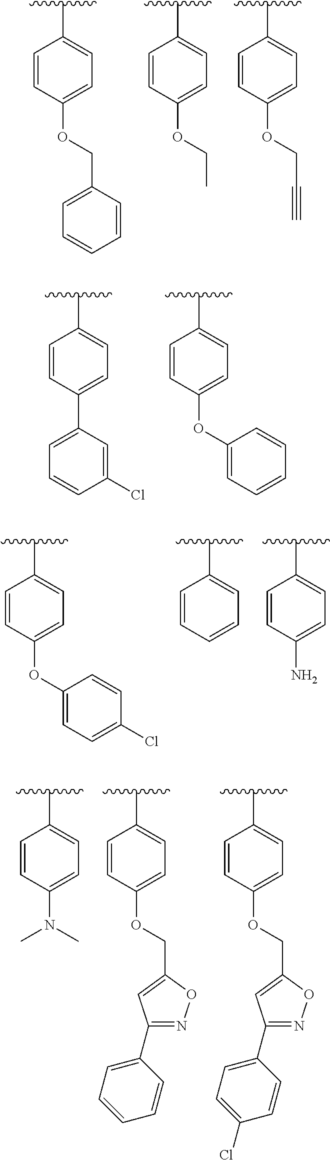

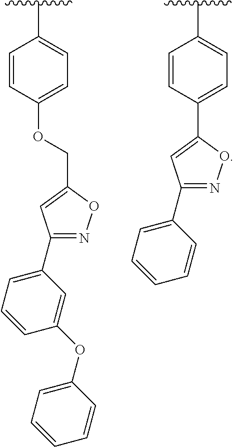 [SMILES: *c1ccc(OCc2cc(-c3cccc(Oc4ccccc4)c3)no2)cc1 ; *c1ccc(-c2cc(-c3ccccc3)no2)cc1]

14. Method according to claim 13, wherein the ligand comprises a moiety of formula (III)

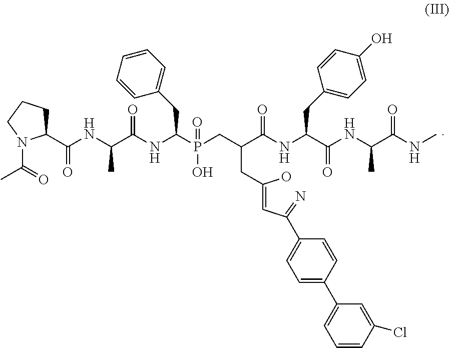 [SMILES: CNC(=O)[C@@H](C)NC(=O)[C@H](Cc1ccc(O)cc1)NC(=O)C(C/C(O)=C/C(=N)c1ccc(-c2cccc(Cl)c2)cc1)CP(=O)(O)[C@H](Cc1ccccc1)NC(=O)[C@@H](C)NC(=O)[C@@H]1CCCN1C(C)=O]

15. Method according to claim 1 for the detection of MMP12, MMP2, or MMP9, preferably MMP12.

16. A kit for specifically detecting an MMP of interest solely in its active form, comprising
an antibody specific for the MMP of interest;
a ligand of the MMP of interest, comprising a phosphonic pseudopeptide inhibitor, preferably functionalizing a carrier protein;
optionally, a solid support on which is immobilized either the antibody or the ligand; and
optionally, reagents enabling detection of the antibody or ligand.

17. (canceled)
